(19) United States
(12) Patent Application Publication
(10) Pub. No.: US 2023/0414779 A1
(43) Pub. Date: Dec. 28, 2023

(54) ANTIGEN-BINDING FRAGMENTS AND USES THEREOF

(71) Applicants: NewSouth Innovations Pty Limited, Sydney, New South Wales (AU); NSW Health Pathology, Westmead, New South Wales (AU)

(72) Inventors: Beng Hock Chong, Sydney, New South Wales (AU); Jose Perdomo, Sydney, New South Wales (AU); Halina Leung, Sydney, New South Wales (AU); Zohra Ahmadi, Sydney, New South Wales (AU)

(21) Appl. No.: 18/247,381

(22) PCT Filed: Sep. 30, 2021
(86) PCT No.: PCT/AU2021/051150

§ 371 Date: Mar. 30, 2023

(30) Foreign Application Priority Data
Sep. 30, 2020 (AU) 2020903540

(51) Int. Cl. A61K 47/68; C07K 16/28; A61P 7/02

(52) U.S. Cl. CPC A61K 47/6849; C07K 16/283; A61K 47/6803; A61P 7/02; C07K 2317/622

(57) ABSTRACT

The present invention provides humanized antibody fragment (scFv) from murine clone IV.3 scFv that specifically binds FcyRIIA, with a linker between the VH and VL with the amino acid sequence W A W V WLTET A V, which linker also binds FcyRIIA, and methods for the use thereof. In certain forms, the present invention provides molecules which may find application in the treatment of thrombogenic and FcyRIIA related diseases and disorders related to the activation of immune complexes.

### INCORPORATION BY CROSS-REFERENCE

The present invention claims priority from Australian provisional patent application number 2020903540, filed on 30 Sep. 2020, the entire content of which is incorporated herein by cross-reference.

### TECHNICAL FIELD

The present invention relates generally to the fields of immunology and medicine. More specifically, the present invention relates to molecules which specifically bind FcγRIIA. In certain forms, the present invention provides molecules which may find application in the treatment and prevention of diseases and disorders related to the activation of immune complexes.

### BACKGROUND

The following discussion of the background of the invention is merely provided to aid the reader in understanding the invention and is not admitted to describe or constitute prior art to the invention.

Thrombosis is one of the main causes of global morbidity and mortality. A prominent example is heparin-induced thrombocytopenia (HIT), a limb- and life-threatening complication of heparin therapy that affects 1-5% of patients receiving unfractionated heparin. HIT is the result of immune reactions to unfractionated (UF) or low molecular weight (LMW) heparin. The reaction is primarily mediated by the HIT antibody, an IgG antibody against the heparin-platelet factor 4 (PF4) complex. Formation of the antibody-antigen complex leads to strong platelet activation and thrombus formation. The immune complex also induces platelet-neutrophil interaction and NETosis. These potent prothrombotic processes (platelet activation, platelet-neutrophil interaction and NETosis) drive thrombosis in HIT. Not surprisingly, the thrombosis in HIT is often extensive and severe, leading to devastating clinical sequelae such as life-threatening arterial thrombosis (e.g. heart attacks and strokes), lung clots (pulmonary emboli), leg gangrene, limb loss, multi-organ failure and death. HIT is relatively common as UF and LMW heparins are widely used in clinical practice. Because of the devastating clinical sequalae, it is a drug complication dreaded by many clinicians globally.

The initial step in treating HIT is the withdrawal of heparin. However, this does not stop the strong thrombotic processes and therefore does not prevent serious clinical sequelae such as limb gangrene and thrombotic deaths. Anticoagulant treatments for HIT are only partially effective in treating thrombosis in HIT as, without extinguishing the initiating/driving events, the use of an anticoagulant alone to inhibit the downstream coagulation pathway events has proven to be inadequate. Anticoagulants are capable of moderately reducing thrombotic events but fail to significantly reduce limb gangrene and mortality rates. Anticoagulant therapy alone does not suppress or extinguish the HIT antibody-induced platelet activation and thrombin generation which together drive thrombosis in HIT.

HIT-like conditions with similarly strong thrombotic processes occur in infections (bacterial, viral—including COVID-19—and fungal) and in autoimmune diseases (including systemic lupus erythematosus, etc.). In these conditions, the antibodies against microorganisms and autoantibodies against specific antigens form immune complexes (ICs), as in HIT, and activate immune cells (monocytes, neutrophils, platelets) (via Fc gamma receptors) and endothelial cells. IC-mediated activation induces neutrophil extracellular traps (NETosis), monocyte ETosis, platelet aggregation, fibrin deposition and thrombus formation. This results in occlusion of vessels of various organs causing tissue damage, morbidity and mortality.

Immune thrombocytopenias (ITPs) are typically conditions that are mediated by pathogenic IgG antibodies that have specificity for platelet receptors GPIIb-IIIa or GPIb-IX. In some patients, these antibodies can activate platelets via the activation of immune complexes and cause thrombosis. ITPs are generally autoimmune diseases with unknown causes or which are secondary to other diseases or disorders, for example, systemic lupus erythematosus, immune reactions to drugs (i.e. drug-induced ITPs) or infection (infection-related ITPs, e.g. ITP associated with human immunodeficiency virus (HIV), Epstein Barr virus (EBV), measles and other infections).

ITPs mediated by anti-GPIb-IX IgG antibodies are often very severe and are refractory to treatment with conventional immunosuppressive therapies. First line treatments include glucocorticosteroids, IVIg, anti-D, or any combination thereof. Second line treatments consist of azathioprine, cyclophosphamide, danazol, vinca alkaloids, rituximab, TPOR-agonists, splenectomy etc. With the exception of TPOR-agonists, anti-GPIb-IX IgG antibody mediated ITP does not respond well to conventional immunosuppressive therapies. Even with TPOR-agonists, it typically takes up to 2 weeks for the platelet response to occur, as these drugs act by stimulating megakaryocyte precursors (platelet producing cells) in bone marrow and about 2 weeks is required for megakaryocyte precursors to mature to a stage where they can produce platelets. If a patient bleeds during this time, there is no effective treatment if the patient has already become refractory to immunosuppressive drugs. Even with TPOR-agonists, 20-30% of patients fail to respond. Third line treatments include combined therapy (combinations of first and second line agents), combined chemotherapy and allogenic bone marrow transplantation. First-, second- and third-line treatments all have serious adverse effects and are not well tolerated. The reasons for the lack of response to therapy of ITPs, specifically those mediated by anti-GPIb-IX antibodies, is currently unknown.

Current drug therapies have not been effective in controlling thrombosis in HIT and ITPs because they fail to extinguish potent prothrombotic processes such as the activation of immune complexes and/or platelets. Molecules that specifically bind to immune complexes and/or platelets are a potential solution to this problem. However, a need exists to find better ways of increasing the binding affinity of such molecules. Portions of molecules, for, example, portions of antibodies, are often combined with linker peptides to specifically bind and block the activity of other biological molecules, for example, immune complexes and/or platelets. The linker peptides are designed to merely allow the portions of molecules, for example, the heavy and light chains of antibodies, to position themselves to access their target. To date, none of these molecules have been capable of efficiently preventing or extinguishing prothrombotic processes.

There is a need for more effective treatments for diseases and disorders related to activation by immune complexes, for example, HIT, HIT-like conditions (viral and bacterial infections) and autoimmune diseases. A need also exists for improving the binding affinity of therapeutic molecules comprising linkers to their biological targets.

### SUMMARY OF THE INVENTION

The present invention addresses at least one of the problems associated with current therapies and/or methods for the prevention of diseases and disorders related to the activation of immune complexes such as HIT, VITT, infections (including COVID-19) and autoimmune diseases.

The present inventors have found that the initiating and/or driving events in HIT and many ITPs may be prevented and/or extinguished by molecules which block antibody receptors on platelets, neutrophils and and/or monocytes, specifically the human Fc gamma receptor IIA (FcγRIIA or CD32a). This receptor has high affinity for immunoglobulin ICs, and upon interaction induces platelet, neutrophil and/or monocyte activation, leading to thrombosis and thrombocytopenia (platelet destruction). ICs are formed in autoimmune diseases and also as a result of bacterial and viral infections.

The present invention provides antigen-binding fragments which bind to and specifically block FcγRIIA which include a weak FcγRIIA-binding peptide inserted as a linker. To avoid adversely interfering with the activity of the molecules, linker peptides are typically of standard lengths and include amino acids with neutral side chains. The present inventors have unexpectedly observed an enhanced binding effect by inserting a functional FcγRIIA-binding peptide within a linker positioned between heavy and light antibody chains. This finding was surprising as the presence of a functional peptide between the heavy and light antibody chains can typically be expected to interfere with and/or reduce the activity of the complementarity determining regions (CDRs) of the heavy and light chains and thereby reduce their binding specificity. Reflecting this, functional peptide/s have traditionally been inserted into the C- or N-termini of antigen-binding fragments so that they do not interfere with the function of the heavy and light chains. As a functional peptide is normally more likely to be exposed at the C- or N-termini, placement at these positions is normally expected to provide maximum functionality.

By placing the FcγRIIA-binding peptide between the heavy and light chains of the antigen-binding fragment, the inventors have also kept the C- and N-termini free for the placement of other functional molecules, for example, anticoagulants. The inventors have also unexpectedly identified that conjugating anticoagulant molecule/s to antigen-binding fragments comprising an FcγRIIA-binding peptide linker significantly increases the binding of the antigen-binding fragments to FcγRIIA. This finding was unexpected given that anticoagulants, for example, lepirudin and/or bivalirudin are not known have any role in binding to FcγRIIA or enhancing the capacity of other molecules to bind to FcγRIIA.

The present invention relates at least in part to the following embodiments.

Embodiment 1. An antigen-binding fragment that specifically binds FcγRIIA, wherein the antigen-binding fragment comprises a heavy chain variable region region, a light chain variable region and a linker, and wherein at least a portion of the linker binds FcγRIIA.

Embodiment 2. The antigen-binding fragment according to embodiment 1, wherein the heavy chain variable region and the light chain variable region are joined by the linker.

Embodiment 3. The antigen-binding fragment according to embodiment 1 or embodiment 2, wherein the antigen-binding fragment comprises:

a heavy chain variable region comprising:
a) a heavy chain CDR1 with an amino acid sequence according to SEQ ID NO: 4;
b) a heavy chain CDR2 with an amino acid sequence according to SEQ ID NO: 5;
c) a heavy chain CDR3 with an amino acid sequence according to SEQ ID NO: 6;
and a light chain variable region comprising:
d) a light chain CDR1 with an amino acid sequence according to SEQ ID NO: 7;
e) a light chain CDR2 with an amino acid sequence according to SEQ ID NO: 8; and
f) a light chain CDR3 with an amino acid sequence according to SEQ ID NO: 9.

Embodiment 4. The antigen-binding fragment according to any one of embodiments 1 to 3, wherein the linker comprises the amino acid sequence:
WAWX₁WX₂TETX₃V
and wherein:
X₁ is selected from V or A;
X₂ is selected from L or A; and
X₃ is selected from A or G.

Embodiment 5. The antigen-binding fragment according to any one of embodiments 1 to 4, wherein the linker comprises an amino acid sequence with at least 90% sequence identity to SEQ ID NO: 11.

Embodiment 6. The antigen-binding fragment according to any one of embodiments 1 to wherein the linker comprises an amino acid sequence according to SEQ ID NO: 11.

Embodiment 7. The antigen-binding fragment according to any one of embodiments 1 to 6, wherein the heavy chain variable region comprises an amino acid sequence with at least 90% sequence identity to SEQ ID NO: 15.

Embodiment 8. The antigen-binding fragment according to any one of embodiments 1 to 7, wherein the light chain variable region comprises an amino acid sequence with at least 90% sequence identity to SEQ ID NO: 16.

Embodiment 9. The antigen-binding fragment according to any one of embodiments 1 to 8, wherein:

the heavy chain variable region comprises an amino acid sequence according to SEQ ID NO:15, or a variant of that sequence having 1, 2, or 3 amino acid substitutions in the framework region; and/or

the light chain variable region comprises an amino acid sequence according to SEQ ID NO:16, or a variant of that sequence having 1, 2, or 3 amino acid substitutions in the framework region.

Embodiment 10. The antigen-binding fragment according to any one of embodiments 1 to 9, wherein the position of the functional linker is:
a) within a heavy and/or light chain but not within a CDR; or
b) 1, 2, 3, 4, 5, 6, 7, 8, 9, 10 or more amino acid positions to the left or right of the position according to SEQ ID NO:3.

Embodiment 11. The antigen-binding fragment according to any one of embodiments 1 to 10, wherein the functional linker is not located at the N-terminus or the C-terminus of the antigen-binding fragment.

Embodiment 12. The antigen-binding fragment according to any one of embodiments 1 to 11, wherein the functional linker is positioned so that it is partially or completely exposed on the outside of the tertiary structure of the antigen-binding fragment.

Embodiment 13. The antigen-binding fragment according to any one of embodiments 1 to 12, wherein the functional linker is positioned so that it enhances the capacity of the antigen-binding fragment to bind to FcγRIIA.

Embodiment 14. The antigen-binding fragment according to any one of embodiments 1 to 13, wherein the antigen-binding fragment is conjugated to an anticoagulant.

Embodiment 15. The antigen-binding fragment according to embodiment 14, wherein the anticoagulant is selected from the group consisting of danaparoid, desirudin, tick anticoagulant peptide, factor Xa inhibitors, prothrombin inhibitors, tissue factor inhibitors, FXII inhibitors, danaparoid, bivalirudin, lepirudin, argatroban, and any combination thereof.

Embodiment 16. The antigen-binding fragment according to embodiment 14 or embodiment 15, wherein the anticoagulant is bivalirudin and/or lepirudin.

Embodiment 17. The antigen-binding fragment according to embodiment 16, wherein the antigen-binding fragment comprises an amino acid sequence according to SEQ ID NO:12, or a variant of that sequence having 1, 2, or 3 amino acid substitutions in the framework region.

Embodiment 18. The antigen-binding fragment according to embodiment 16, wherein the antigen-binding fragment comprises an amino acid sequence according to SEQ ID NO:14, or a variant of that sequence having 1, 2, or 3 amino acid substitutions in the framework region.

Embodiment 19. The antigen-binding fragment according to any one of embodiments 1 to 18, wherein the antigen-binding fragment is a single-chain variable fragment (scFv).

Embodiment 20. The antigen-binding fragment according to any one of embodiments 1 to 19, wherein the functional linker is positioned adjacent to or within a flexible linker.

Embodiment 21. The antigen-binding fragment according to any one of embodiments 1 to 20, wherein the flexible linker comprises or consists of neutral amino acids.

Embodiment 22. The antigen-binding fragment according to any one of embodiments 1 to 21, wherein the flexible linker comprises or consists of between 1 and 15 amino acids, between 3 and 14 amino acids, between 3 and 14 amino acids, between 9 and 13 amino acids, between 10 and 12 amino acids, or 11 amino acids.

Embodiment 23. A nucleic acid molecule encoding the antigen-binding fragment according to any one of embodiments 1-22.

Embodiment 24. A vector comprising the nucleic acid molecule according to embodiment 23.

Embodiment 25. A host cell comprising the vector according to embodiment 24.

Embodiment 26. The host cell according to embodiment 25, wherein the host cell is derived from a mammal, insect, plant or microbe.

Embodiment 27. A pharmaceutical composition comprising the anti-binding fragment of any one of embodiments 1-22.

Embodiment 28. A method of treating a subject with a thrombogenic-related disease, the method comprising administering to the subject a therapeutically effective amount of the antigen-binding fragment of any one of embodiments 1-22 or the pharmaceutical composition of embodiment 27.

Embodiment 29. Use of the antigen-binding fragment of any one of embodiments 1-22 in the manufacture of a medicament for treating a thrombogenic-related disease in a subject in need thereof.

Embodiment 30. The antigen-binding fragment of any one of embodiments 1-22 for use in the treatment of a thrombogenic-related disease in a subject in need thereof.

Embodiment 31. The method according to embodiment 28, the use according to embodiment 29 or the antigen-binding fragment according to embodiment 30, wherein the thrombogenic-related disease is heparin-induced thrombocytopenia (HIT), immune thrombocytopenia (ITP), an immune platelet disorder with associated thrombosis, NETs-induced thrombo-embolism, organ injury, a NETs-associated disorder, drug-induced ITP, viral infection (e.g. SARS infection, COVID-19 infection), bacterial infection, fungal infection, parasitic infection, sepsis, antibody-induced ITP, antiphospholipid syndrome, cancer-induced thrombocytopenia, thrombo-embolism, an autoimmune or inflammatory disease involving CD32 (including rheumatoid arthritis, osteoarthritis, systemic lupus erythematosus and psoriasis), or a disorder or disease mediated by CD32 involving one or more of the following cells: platelets, neutrophils, monocytes, macrophages, eosinophils, basophils and mast cells.

Embodiment 32. The method, use or antigen-binding fragment according to embodiment 31, wherein the thrombogenic-related disease involves the binding of immune complexes to FcγRIIA.

Embodiment 33. The method, use or antigen-binding fragment according to embodiment 31 or embodiment 32, wherein the thrombogenic-related disease is heparin-induced thrombocytopenia (HIT).

Embodiment 34. The method, use or antigen-binding fragment according to embodiment 31 or embodiment 32, wherein the ITP is primary ITP with associated thrombosis.

Embodiment 35. The method, use or antigen-binding fragment according to embodiment 31 or embodiment 32, wherein the ITP is secondary ITP.

Embodiment 36. The method, use or antigen-binding fragment according to embodiment 35, wherein the secondary ITP is secondary ITP with associated anti-phospholipid antibody syndrome, systemic lupus erythematosus, Evans syndrome or chronic infection.

Embodiment 37. A method of treating a subject with a disease related to FcγRIIa-mediated neutrophil activation, the method comprising administering to the subject a therapeutically effective amount of the antigen-binding fragment of any one of embodiments 1-22 or the pharmaceutical composition of embodiment 27.

Embodiment 38. Use of the antigen-binding fragment of any one of embodiments 1-22 in the manufacture of a medicament for treating a disease related to FcγRIIa-mediated neutrophil activation in a subject in need thereof.

Embodiment 39. The antigen-binding fragment of any one of embodiments 1-22 for use in the treatment of a disease related to FcγRIIa-mediated neutrophil activation in a subject in need thereof.

Embodiment 40. The method according to any one of embodiments 28 or 31 to 37, the use according to any one of embodiments 29, 31 to 36 or 38, or the antigen-binding fragment according to any one of embodiments 30 to 36 or 39, wherein the antigen-binding fragment is administered by a route selected from the group consisting of intravenous, intramuscular, subcutaneous, intraperitoneal, or any combination thereof.

Embodiment 41. The method according to any one of embodiments 28, 31 to 37 or 40, the use according to any one of embodiments 29, 31 to 36, 38 or 40, or the antigen-binding fragment according to any one of embodiments 30 to 36 or 39 to 40, wherein the amount of antigen-binding fragment administered is from about 5 mg/kg to about 50 mg/kg, or the amount of antigen-binding fragment administered is via intravenous infusion at a dosage of about 0.1 mg/kg/hr to about 0.5 mg/kg/hr, about 0.1 mg/kg/hr to about 1 mg/kg/hr, about 0.5 mg/kg/hr to about 5 mg/kg/hr, or at about 5 mg/kg/hr to about 10 mg/kg/hr.

Embodiment 42. The method according to any one of embodiments 28, 31 to 37 or 40 to 31, the use according to any one of embodiments 29, 31 to 36, 38 or 40 to 41, or the antigen-binding fragment according to any one of embodiments 30 to 36 or 39 to 41, wherein the subject is human.

Embodiment 43. A method of treating a subject with vaccine-induced immune thrombotic thrombocytopenia (VITT), the method comprising administering to the subject a therapeutically effective amount of the antigen-binding fragment of any one of embodiments 1-22 or the pharmaceutical composition of embodiment 27.

Embodiment 44. Use of the antigen-binding fragment of any one of embodiments 1-22 in the manufacture of a medicament for treating VITT in a subject in need thereof.

Embodiment 45. The antigen-binding fragment of any one of embodiments 1-22 for use in the treatment of VITT in a subject in need thereof.

### Definitions

As used in this application, the singular form “a”, “an” and “the” include plural references unless the context clearly dictates otherwise. For example, the term “antigen-binding fragment” also includes multiple antigen-binding fragments.

As used herein, the term “comprising” means “including”, in a non-exhaustive sense. Variations of the word “comprising”, such as “comprise” and “comprises” have correspondingly varied meanings. Thus, for example, an antigen-binding fragment “comprising” SEQ ID A and SEQ ID B may consist exclusively of SEQ ID A and SEQ ID B, or may include one or more additional components such as SEQ ID C.

As used herein, the term “between” when used in reference to a range of numerical values encompasses the numerical values at each endpoint of the range.

As used herein, the term “about”, when used in reference to a recited numerical value, includes the recited numerical value and numerical values within plus or minus ten percent of the recited value.

As used herein, the term “plurality” means more than one. In certain specific aspects or embodiments, multiple may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or more, and any numerical value derivable therein, and any range derivable therein.

As used herein, the terms “protein”, “peptide” and “polypeptide” each refer to a polymer made up of amino acids linked together by peptide bonds and are used interchangeably. For the purposes of the present invention a “polypeptide” may constitute a full-length protein or a portion of a full-length protein.

As used herein, the term “isolated”, when used in reference to a biological molecule (e.g. an antigen-binding fragment), refers to a biological molecule that is free from at least some of the components with which it naturally occurs.

As used herein, the terms “antibody” and “antibodies” will be understood to mean an antibody made up of two heavy chains and two light chains with both Fc regions and Fab regions. Antibodies may be of IgG (including IgG1, IgG2, IgG3, and IgG4), IgA (including IgA1 and IgA2), IgD, IgE and IgM isotypes.

As used herein, the term “antigen-binding fragment” includes, but is not limited to, Fv, Fab, Fab′, F(ab′)2, Fd, single-chain Fv (scFv), single-chain antibody, disulphide-linked Fv (sdFv) and fragments comprising either a VL or VH domain.

As used herein, the term “linker” refers to a peptide which joins a heavy chain and a light chain of an antigen-binding fragment.

As used herein, the term “functional linker” will be understood to mean a linker which binds to the target molecule to which an antigen-binding fragment specifically binds.

As used herein, the term “humanized” when used in reference to an antigen-binding fragment or antibody will be understood to mean an antigen-binding fragment or antibody from a non-human species containing modification/s to its protein sequence which increase its similarity to a human sequence.

As used herein, the term “single chain variable fragment” or “scFv” will be understood to mean a polypeptide comprising heavy and light antibody chains joined by a linker.

As used herein, the terms “binding specificity” and “specifically binding”, when used in reference to antibody, antibody variant, antibody derivative, antigen-binding fragment, and the like refer to its capacity to bind a given target molecule preferentially over other non-target molecules. For example, if the antibody, antibody variant, antibody derivative, or antigen-binding fragment (“molecule A”) is capable of “binding specifically” or “specifically binding” to a given target molecule (“molecule B”), molecule A has the capacity to discriminate between molecule B and any other number of potential alternative binding partners. Accordingly, when exposed to multiple different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding molecules that are not molecule B at a weak, yet detectable level. This is commonly known as background binding and is readily discernible from molecule B-specific binding, for example, by use of an appropriate control.

As used herein, “FcγRIIA”, also known in the art as “CD32a” or “the Fc fragment of IgG receptor Ha” will be understood to mean a cell surface receptor found on phagocytic cells such as macrophages and neutrophils which is involved in the process of phagocytosis and clearing of immune complexes.

As used herein, the terms “treat”, “treating”, “treatment”, and the like refer to reducing or ameliorating a disorder/disease and/or symptoms associated therewith. It will be appreciated, although not precluded, that treating a disorder or condition does not require that the disorder, condition, or symptoms associated therewith be completely eliminated.

As used herein, the term “subject” refers to any animal (e.g., a mammal), including, but not limited to, humans, non-human primates, canines, felines, and rodents.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, an amino acid sequence (“subject sequence”) having at least 95% “sequence identity” with another amino acid sequence (“query sequence”) is intended to mean that the subject sequence is identical to the query sequence except that the subject sequence may include up to five amino acid alterations per 100 amino acids of the query sequence. In other words, to obtain an amino acid sequence of at least 95% sequence identity to a query sequence, up to 5% (i.e. 5 in 100) of the amino acids in the subject sequence may be inserted or substituted with another amino acid or deleted.

### BRIEF DESCRIPTION OF THE FIGURES

Preferred embodiments of the present invention will now be described by way of example only, with reference to the accompanying figures wherein:

FIG. 1A provides Western blots showing the purified proteins obtained from bacterial expression. Arrows indicate the protein band corresponding to each HRU molecule. Molecular weight markers (m) are also shown.

FIG. 1B provides representative flow cytometry histograms of AF488-labelled HRU molecules binding to human platelets. Histogram labels: filled: negative control, open: AF488-labelled HRU scFvs.

FIG. 1C provides a graph showing the percentage of positive cells (platelets) bound by HRU scFvs at different concentrations as determined by flow cytometry.

FIG. 1D provides graphs of platelet-based fluorescent assay measurements with K_{D} and R² values for the parental scFv, HRU5 and HRU6. Non-linear regression analysis (one-site specific binding) was used to determine K_{D} and R² values. GMF, geometric mean fluorescence.

FIG. 2A provides representative platelet aggregation traces. Platelet aggregation was induced with HIT patient's serum in the absence (HIT serum only), or presence of inhibitory amounts of parental scFv, HRU5 or HRU6 as indicated by the arrows. The y axis indicates percentage of platelet aggregation.

FIG. 2B provides representative platelet aggregation traces. Platelets were treated as in FIG. 2A, using partially inhibiting concentrations of parental scFv (5.2 nM), HRU5 (5.2 nM) or HRU6 (4.3 nM) to illustrate time delays which provide more sensitive and reliable estimation of the inhibition of IC-induced platelet aggregation than the decrease in extent of platelet aggregation. Arrows on top of the graph indicate time delay (i.e., time lapsed before platelets start aggregating).

FIG. 2C provides time delay curves. Non-linear regression analysis (sigmoidal dose-response curves) was used to determine the effective concentration, 50% (EC50) for parental scFv, HRU5 and HRU6.

FIG. 2D provides inhibition of platelet aggregation curves. Non-linear regression analysis (sigmoidal dose-response curves) was used to determine the inhibitory concentration, 50% (IC50) for parental scFv, HRU5 and HRU6.

FIG. 3A provides graphs depicting reconstitution of the HIT condition using whole blood in a microfluidics chamber. The graphs show the percentage area coverage (coverage of the microchannels) of thrombus components (DNA, neutrophils and platelets) versus time (minutes) with and without parental scFv, HRU5, HRU6 or HRU7.

FIG. 3B is a graph of a Thrombin Time Delay assay for HRU6 and HRU7. Increasing concentrations of HRU molecules were incubated with undiluted plasma prepared from ACD anticoagulated blood. Clotting time of control plasma (untreated plasma) is shown corresponding to 0 concentration (closed circle). HRU5 does not contain an anticoagulant peptide and does not prolong clotting time (only highest dose is shown, square). HRU7 (solid line) and HRU6 (dotted line) prolong clotting time in a dose-dependent manner. Clotting time is shown in sec.

FIG. 3C provides fluorescence microscopy images of fibrin deposition. Whole blood was incubated with 0.08 U/ml of thrombin and stained with Alexa Fluor 647 anti-fibrin monoclonal antibody in the absence (vehicle) or presence of HRU5 or HRU6. White fluorescence indicates fibrin deposition. HRU6 completely inhibits fibrin deposition.

FIG. 4 is a graph of the serotonin release assay. Serotonin release was induced by a HIT-like monoclonal antibody (KKO) and was inhibited by parental scFv, HRU5, HRU6 or HRU7. Heparin was used at therapeutic low concentrations (L, 0.1 U) and at inhibitory high concentration (H,100 U). Dotted lines denote 0% and 20% CPMA. Values over 20% are considered positive. CPMA, counts per minute.

FIG. 5A provides images of inhibition of thrombosis in mice. Mouse platelets were labelled in vivo with anti CD42c-Dylight649 antibody. Animals were treated with HIT-like antibody KKO in the absence (vehicle) or presence of parental scFv or HRU5. Fixed lungs were scanned on an IVIS Lumina Spectrum CT scanner. Fluorescence in the lungs (represented as lighter areas) indicates the presence of thrombi.

FIG. 5B is a graphical representation of fluorescence intensity of the lungs shown in FIG. 5A.

FIG. 6A is a plot showing the binding of HRU4, HRU5 and HRU6 to monocytes in vivo. Labelled HRU proteins were injected intravenously into double transgenic mice (FcγRIIA/hPF4). Mice were bled at 1 min and 15 min and binding was determined by flow cytometry.

FIG. 6B provides a graphical representation of the data shown in FIG. 6A.

FIG. 7 is a plot showing the binding of HRU4, HRU5 and HRU6 to human platelets. Platelets were incubated with labelled HRU proteins at different concentrations. Binding was analysed by flow cytometry. GMF is geometric mean fluorescence.

FIG. 8 is a graph of radiant efficiency showing the level of platelet accumulation in FcγRIIa+/hPF4+ mouse lungs. Administration of HRU4 led to strong inhibition of thrombosis.

FIG. 9 provides plots of raw data obtained by inhibiting platelet aggregation induced by serum from a HIT patient which was used to calculate EC50. EC50 was calculated by fitting a curve to the data as shown. Inhibition of platelet aggregation of HIT-antibody by HRU5 ˜3.5 mg/ml.

FIG. 10 provides plots comparing the effect of the FcgRIIA binding peptide and HRU5 on inhibiting platelet aggregation induced by serum from a HIT patient. Inhibition of platelet aggregation of HIT-antibody by HRU5 ˜3.5 mg/ml.=˜93.86 nM. Inhibition of platelet aggregation of HIT-antibody by the FcgRIIA binding peptide was at 100 μM.

FIG. 11 provides a graph of the IC50 for the FcgRIIA binding peptide.

FIG. 12 is a graph comparing the IC50 of the C1 construct to that of HRU5.

FIG. 13 is a model of the Parental scFv. The CDRs within the VL and VH regions and the linker are indicated.

FIG. 14 is a model of HRU5. The CDRs within the VL and VH regions and the linker are indicated.

FIG. 15 is a plot of the results of gel filtration of purified C1 (peptide at the N-terminus-HRU4) loaded in the gel filtration column—an extremely low level of the desired protein was obtained at 24-28 ml.

FIG. 16 is a plot of the results of a second gel filtration of purified C1 obtained from 24 ml of bacterial culture.

FIG. 17 is a plot of the results of gel filtration of purified HRU5 (Peptide in the linker) loaded in the gel filtration column. A detectable peak can be observed.

FIG. 18 is a plot of the results of a second gel filtration of purified HRU5 obtained from 3 L of bacterial culture.

FIG. 19 is a graph showing the expression of C1 and HRU5 in E. coli.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

Heparin-induced thrombocytopenia (HIT) is the result of immune reactions to unfractionated (UF) or low molecular weight (LMW) heparin. The reaction is primarily mediated by the HIT antibody, an IgG antibody against the heparin-platelet factor 4 (PF4) complex. Formation of the antibody-antigen complex leads to strong platelet activation and thrombus formation. The immune complex also induces platelet-neutrophil interaction and NETosis. These potent prothrombotic processes (platelet activation, platelet-neutrophil interaction and NETosis) drive thrombosis in HIT.

Immune thrombocytopenias (ITPs) are typically conditions that are mediated by pathogenic IgG antibodies that have specificity for platelet receptors GPIIb-IIIc or GPIb-IX. In some patients, these antibodies can activate platelets via activation of immune complexes and cause thrombosis.

Without wishing to be bound by theory, it is postulated that the present invention works by providing antigen-binding fragments which block the FcγRIIa receptor, thereby preventing thrombus formation. The present invention provides antigen-binding fragments which bind to and specifically block FcγRIIA which include a weak FcγRIIA-binding peptide inserted as a linker. Customarily, “linker” peptides are of restricted lengths and with particular physical properties such as flexibility and non-interference with the scFv structure. The present inventors have surprisingly found an additive functional effect by inserting a functional FcγRIIA-binding peptide in the linker position between heavy and light antibody chains, which also bind FcγRIIA. This result is certainly not obvious and is also surprising as the presence of an unknown peptide (not traditionally used as a linker) in between the heavy and light antibody chains would normally be expected to interfere with and/or reduce the activity of the complementarity determining regions (CDRs) of the heavy and light chains and thereby reduce binding specificity unless this peptide is known not to obstruct the antigen binding of the CDRs or not to alter the tertiary structure of the scFv or to be present in an accessible position when inserted as a linker (for it to retain FcγRIIA binding function as traditional linker peptides usually occur in hidden positions within the protein structure). There is no published or publicly known data that this peptide has these properties. Accordingly, functional peptide/s have traditionally been inserted into the C- or N-termini of antigen-binding fragments so that they do not interfere with the function of the heavy and light chains and so that the functional peptide is more likely to be exposed for maximum functionality. Inserting the functional peptide in the particular linker position of the scFV is an unconventional, innovative step with unpredictable results; its positive outcomes observed are not obvious and unpredictable given the above constraints.

By placing the FcγRIIA-binding peptide between the heavy and light chains of the antigen-binding fragment, the inventors have freed the C- and N-termini for the placement of other functional molecules, for example, anticoagulants. The inventors of the present invention have further surprisingly found that conjugating the antigen-binding fragments comprising the FcγRIIA-binding peptide as a linker to anticoagulant molecule/s greatly increases the binding of the antigen-binding fragments to FcγRIIA. This finding was very surprising as anticoagulants, for example, lepirudin and/or bivalirudin are not known to bind FcγRIIA.

Accordingly, certain embodiments of the present invention provide FcγRIIa-specific antigen-binding fragments and pharmaceutical compositions comprising antigen-binding fragments and variants thereof. Other embodiments of the present invention relate to methods of treating diseases and disorders associated with immune complex activation, including thrombogenic-related diseases, in subjects afflicted with the same. Further aspects of the present invention relate to medicaments comprising antigen-binding fragments. Also contemplated by the present invention are antigen-binding fragments for use in methods of treating thrombogenic-related diseases.

### Antigen-Binding Fragments

The present invention provides antigen-binding fragments, plus methods, pharmaceutical compositions and medicaments comprising at least one FcγRIIa-specific antigen-binding fragment, derivative or variant thereof.

An FcγRIIa-specific antigen-binding fragment according to the invention may comprise a heavy chain and a light chain which may or may not be derived from the same source.

The antigen-binding fragments and/or variants thereof according to the present invention are not restricted to any particular isotype. In some embodiments, they are humanized antigen-binding fragments and/or variants thereof.

Included within the scope of the present invention are “fragments” of antibodies. In general, the fragments are “antigen-binding fragments” in the sense that they are capable of specifically binding to an antigen and/or epitope (e.g. FcγRIIa) as was the parent antibody from which they are derived or upon which they are based. Typically, an antigen-binding fragment retains at least 10% of the antigen and/or epitope binding capacity of the parent antibody, or, at least 25%, 50%, 60%, 70%, 80%, 90%, 95%, 99% or 100% of the antigen and/or epitope binding capacity of the parent antibody. It is also contemplated that an antigen-binding fragment of an antibody described herein may include conservative amino acid substitutions that do not substantially alter its antigen and/or epitope binding specificity and/or capacity (e.g. at least 70%, 80%, 90%, 95%, 99% or 100%, of its antigen and/or epitope binding specificity and/or capacity may be retained).

Non-limiting examples of antigen-binding fragments include portions of a full-length antibody, peptide and derivatives thereof including, for example, Fab, Fab′, F(ab)2, F(ab)3, Fv, single-chain Fc (scFv), dsFv, Fd fragments, Fab fragment molecules (e.g. scFv), minibodies, diabodies, triabodies, tetrabodies, kappa bodies, linear antibodies, multispecific antibody fragments formed from antibody fragments, and any portion or peptide sequence of the antibody that is capable of specifically binding to the relevant antigen and/or epitope (e.g. FcγRIIa). A further non-limiting example includes a single-chain antigen-binding fragment comprising a heavy chain variable region and a light chain variable region connected by a linker, wherein the antigen-binding fragment regions may be homologous or heterologous. An even further non-limiting example includes a single-chain antigen-binding fragment comprising a heavy chain variable region and a light chain variable region linked by a functional linker.

An antigen-binding fragment may refer to an scFv comprising a heavy chain and a light chain joined by a linker. Antigen-binding fragments or parts/components thereof may be derived from any animal origin or appropriate production host. Antigen-binding fragments, including single-chain antibodies, may comprise the variable region/s alone or in combination with the entire or part of the following: hinge region, CH1, CH2, and/or CH3 domains. Also included is any combination of variable region/s and hinge region/s, CH1, CH2, and CH3 domains. Antigen-binding fragments may be monoclonal, polyclonal, chimeric, humanized, and human monoclonal and polyclonal antibodies.

An antigen-binding fragment of the present invention may comprise any one or more of SEQ ID NO: 4, SEQ ID NO: 5, SEQ ID NO: 6, SEQ ID NO: 7, SEQ ID NO: 8 and SEQ ID NO: 9. In some embodiments, the antigen-binding fragment may comprise a heavy chain CDR1 with an amino acid sequence according to SEQ ID NO: 4; and/or a heavy chain CDR2 with an amino acid sequence according to SEQ ID NO: 5; and/or a heavy chain CDR3 with an amino acid sequence according to SEQ ID NO: 6; and/or a light chain CDR1 with an amino acid sequence according to SEQ ID NO: 7; and/or a light chain CDR2 with an amino acid sequence according to SEQ ID NO: 8; and/or a light chain CDR3 with an amino acid sequence according to SEQ ID NO: 9. In further embodiments, the heavy chain and light chain variable regions are joined by a functional linker. The functional linker may comprise or consist of an amino acid sequence according to SEQ ID NO: 11.

In further embodiments of the invention, an antigen-binding fragment may comprise a heavy chain CDR1 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 4; and/or a heavy chain CDR2 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 5; and/or a heavy chain CDR3 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 6; and/or a light chain CDR1 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 7; and/or a light chain CDR2 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 8; and/or a light chain CDR3 with an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 9.

In some embodiments of the invention, the heavy chain and light chain variable regions are joined by a functional linker. At least a portion of the functional linker may bind FcγRIIA. The functional linker may comprise the amino acid sequence WAWX₁WX₂TETX₃V, wherein: X₁ is selected from V or A; X₂ is selected from L or A; and X₃ is selected from A or G. The functional linker may comprise or consist of an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 11.

The heavy chain variable region of the antigen-binding fragment of the invention may comprise or consist of an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 15. The light chain variable region of the antigen-binding fragment of the invention may comprise or consist of an amino acid sequence with at least 85%, at least 90%, at least 91%, at least 92%, at least 93%, at least 94%, at least 95%, at least 96%, at least 97%, at least 98%, at least 99%, or 100% sequence identity to SEQ ID NO: 16. In some embodiments of the invention, the heavy chain variable region comprises an amino acid sequence according to SEQ ID NO:15, or a variant of that sequence having 1, 2, or 3 amino acid substitutions in the framework region; and/or the light chain variable region comprises an amino acid sequence according to SEQ ID NO:16, or a variant of that sequence having 1, 2, or 3 amino acid substitutions in the framework region.

The antigen-binding fragments of the present invention may be conjugated to one or more anticoagulants. Non-limiting examples of suitable anticoagulants include danaparoid, desirudin, tick anticoagulant peptide, factor Xa inhibitors, prothrombin inhibitors, tissue factor inhibitors, FXII inhibitors, danaparoid, bivalirudin, lepirudin and argatroban.

The functional linker may be positioned adjacent to or within a flexible linker. In some embodiments, the flexible linker may comprise or consist of neutral amino acids. The length of the flexible linker could be 1, 2, 3, 4, 5, 6, 7, 8, 9, 10, 11, 12 13, 15 or more amino acids. In some embodiments of the invention, the flexible linker comprises or consists of between 1 and 15 amino acids, between 3 and 14 amino acids, between 9 and 13 amino acids, between 10 and 12 amino acids, or 11 amino acids. Although the antigen-binding fragments in the Examples provided herein include a flexible linker, certain embodiments include antigen-binding fragments in which the functional linker is only flanked on one side by a flexible linker. In yet other embodiments, the antigen-binding fragments do not include a flexible linker.

Specific examples of antigen-binding fragments specific for FcγRIIa according to the present invention include the scFvs HRU5 to HRU7.

HRU5 (comprising an amino acid sequence as set forth in SEQ ID NO: 3) is an scFv derived from the mouse IV.3 monoclonal antibody (moAb) constructed by joining single variable heavy chain and light chain domains of the IV.3 antibody with a GGGGWAWVWLTETAVGGGGS linker. HRU5 has undergone some mutations in the framework regions when compared to IV.3. HRU6 and HRU7 comprise HRU5 conjugated to lepirudin (HRU6; comprising an amino acid sequence as set forth in SEQ ID NO: 12) and bivalirudin (HRU7; comprising an amino acid sequence as set forth in SEQ ID NO: 14).

Anticoagulant/s may be conjugated to the antigen-binding fragments of the invention using a peptide linker. The linker used to connect the molecules may be any suitable linker for use in protein chemistry as known in the art. The linker may be substantially linear in nature or may be branched. In certain embodiments, the linker may comprise an oligopeptide, or may be peptidomimetic (comprising a number of, or only amino acid analogues). The linker may be derived from a native sequence, a variant thereof, or a synthetic sequence. Oligopeptide or peptidomimetic linkers may comprise naturally occurring or non-naturally occurring amino acids, or a combination of both. A non-limiting example of a linker for use in the antigen-binding fragments according to the present invention may comprise an oligopeptide of between amino acids, which may, for example, comprise a regular series of glycine and 1 to 3 serines to enhance solubility.

In some embodiments of the invention, the functional linker is positioned on the outside of the tertiary structure of the antigen-binding fragment so that it is exposed and/or partially or wholly accessible for binding to FcγRIIa.

It will be appreciated by those skilled in the art that the functional linker could be positioned 1, 2, 3, 4, 5 or 6 amino acid positions to the left of the position shown in HRU5, HRU6 and/or HRU7 of the Examples in the specification and still provide the benefits of the invention. It will also be appreciated that the functional linker could be positioned 1, 2, 3, 4 or amino acid positions to the right of the position shown in HRU5 HRU6 and/or HRU7 of the Examples in the specification and still provide the benefits of the invention. The functional linker could be positioned within the heavy chain of the light chain of the antigen-binding fragments, but not within the CDRs.

In some embodiments of the invention, the functional linker may be positioned 1, 2, 3, 4, 5, 6, 7, 8, 9, 10 or up to 28 amino acid positions to the left or right of the position shown in HRU5 of the Examples in the specification.

Also included within the scope of the invention are “derivatives” of antigen-binding fragments described herein. A “derivative” of an antigen-binding fragment of the present invention refers to an antigen-binding fragment described herein that is modified to incorporate additional components or have existing component/s altered, but is still capable of specifically binding to the same antigen and/or epitope (e.g. FcγRIIa) as the parent antibody from which it is derived. Typically, an antigen-binding fragment derivative as contemplated herein retains at least 10% of the antigen/epitope binding capacity of the parent antibody, or, at least 25%, 50%, 60%, 70%, 80%, 90%, 95%, 99% or 100% of the antigen and/or epitope binding capacity of the parent antibody from which it derived.

Non-limiting examples of modifications suitable to form antibody derivatives include amidation, glycosylation, phosphorylation, pegylation, lipidation, linage to a cellular ligand or other protein, derivatization by known protecting/blocking groups, acetylation, and the like.

Additionally or alternatively, the derivative may contain one or more non-classical amino acids.

The antigen-binding fragment derivatives may be formed from covalent modification of the antigen-binding fragment described herein, for example, by reacting targeted amino acid residues of the antigen-binding fragment with an agent capable of reacting with selected side chains or terminal residues. For example, derivatization with bifunctional agents is a useful means for cross-linking antigen-binding fragments to macromolecular carriers such as water-insoluble support matrices. Antigen-binding fragments and derivatives thereof as contemplated herein may have an agent attached to the antigen-binding fragment capable of increasing its half-life in vivo (e.g. extending the length of time before clearance from the blood stream). A non-limiting example of such a technique includes the addition of PEG moieties.

In certain embodiments, the antigen-binding fragment derivative may be a multimer, such as, for example a dimer, comprising one or more monomers, where each monomer includes (i) an antigen binding region as described herein, or a polypeptide region derived therefrom (such as, for example, by conservative substitution of one or more amino acid/s), and (ii) a multimerizing (e.g. dimerizing) polypeptide region, such that the antigen-binding fragment forms multimers (e.g. homodimers) that specifically bind to antigens and/or epitopes (e.g. FcγRIIa). For example, an antigen-binding region of anti-FcγRIIa antigen-binding fragment as described herein or polypeptide region derived therefrom, may be recombinantly or chemically fused with a heterologous protein, wherein the heterologous protein comprises a dimerization or multimerization domain. The derivative may be subjected to conditions allowing formation of a homodimer or heterodimer. The heterodimer may comprise identical dimerization domains but different anti-FcγRIIa antigen-binding fragments, identical anti-FcγRIIa antigen-binding fragments but different dimerization domains, or different anti-FcγRIIa antigen-binding fragments and different dimerization domains. Suitable dimerization domains include those that originate from transcription factors (e.g. basic region leucine zipper and zinc fingers), a basic-region helix-loop-helix protein, and an immunoglobulin constant region (e.g. heavy chain constant region or domain thereof such as a CH1 domain, a CH2 domain, or a CH3 domain).

Included within the scope of the present invention are “variants” of the antigen-binding fragments described herein. A “variant” antigen-binding fragment refers to an antigen-binding fragment which alters the amino acid sequence from a “parent” antigen-binding fragment amino acid sequence by virtue of addition, deletion, and/or substitution of one or more amino acid residue/s in the parent antigen-binding fragment sequence. For example, the variant antigen-binding fragment may comprise one or more amino acid substitution/s in one or more CDR and/or framework region/s of the parent antigen-binding fragment (e.g. between 1 and between 2 and 5, or 1, 2, 3, 4 or 5 substitutions in one or more heavy and/or light chain CDR and/or framework regions of the parent antigen-binding fragment). The antigen-binding fragment variant may comprise a heavy chain variable domain sequence and/or a light chain variable domain sequence having at least 50%, at least 60%, at least 70%, at least 80%, at least 85%, at least 90%, at least 95%, or at least 98% amino acid sequence homology (i.e. sequence identity) with the corresponding variable domain of the parent antigen-binding fragment.

Sequence homology or identity between two sequences is defined herein as the percentage of amino acid residues in the candidate sequence that are identical with the parent antigen-binding fragment residues, after aligning the sequences and introducing gaps, if necessary, to achieve the maximum percent sequence identity. If the two sequences which are to be compared with each other differ in length, sequence identity relates to the percentage of amino acid residues of the shorter sequence which are identical with the amino acid residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive, Madison, Wisconsin, 53711) and/or the program “fasta20u66” (version 2.0u66, September 1998 by William R. Pearson and the University of Virginia; see also W. R. Pearson (1990), Methods in Enzymology 183, 63-98).

In some embodiments, a variant antigen-binding fragment as described herein may differ from a parent antigen-binding fragment by way of conservative amino acid change/s in the sequence of variable antigen-binding fragment. A “conservative change” refers to an alteration that is substantially antigenically or conformationally neutral, producing minimal changes in the tertiary structure of the variant antigen-binding fragment, or producing minimal changes in the antigenic determinants of the variant antigen-binding fragment, as compared the parent antigen-binding fragment, and one which does not render the derivative incapable of binding to the same epitope in the respective antigen as the parent antigen-binding fragment. Non-limiting examples of conservative amino acid changes include substitution of hydrophobic amino acids and substitutions of physiochemically similar amino acids. Persons of ordinary skill in the art can routinely and without difficulty assess whether a given amino acid substitution can be made while maintaining conformational and antigenic neutrality (see, for example Berzofsky, (1985) Science 229:932-940; Bowie et al. (1990) Science 247: 1306:1310)

Alterations in protein conformation may be achieved using well-known assays including, but not limited to, microcomplement fixation methods (see Wasserman et al. (1961) J. Immunol. 87:290-295; Levine et al. (1967) Meth. Enzymol. 11:928-936) and through binding studies using conformation-dependent monoclonal antibodies (see Lewis et al. (1983) Biochem. 22:948-952. The conservative amino acid change/s may occur in one or more CDR and/or framework region/s of the parent antigen-binding fragment (e.g. between 1 and 10, between 2 and 5, or 1, 2, 3, 4, or 5 conservative substitutions in one or more CDR and/or framework regions of the parent antigen-binding fragment).

The antigen-binding fragment of the present invention may comprise a humanized derivative of a non-human antigen-binding fragment as described herein. A “humanized” antigen-binding fragment as contemplated herein is a human/non-human chimeric antigen-binding fragment that contains a minimal sequence derived from non-human immunoglobulin. For example, a humanized antigen-binding fragment may be a human immunoglobulin (recipient antibody) in which residues from CDR region/s of the recipient are replaced by residues from a CDR region of a non-human species (donor CDR) (e.g. a mouse, rat, rabbit, or non-human primate having some desired specificity and affinity for an FcγRIIa). Framework region (FR) residues of the human immunoglobulin may also (optionally) be replaced by corresponding non-human residues, and in some cases humanized antigen-binding fragment may comprise residues not present in the recipient antigen-binding fragment or in the donor antigen-binding fragment to enhance the antigen-binding fragment's performance.

Further contemplated herein are “chimeric” antigen-binding fragment derivatives in which a portion of the heavy and/or light chain is identical with or homologous to corresponding sequences of an antigen-binding fragment described herein derived from a particular species or belonging to a particular antibody class or subclass, while the remainder of the chain/s is/are identical with or homologous to corresponding sequences in an antigen-binding fragment derived from another different species or belonging to another different antibody class or subclass. For example, a chimeric antigen-binding fragment as contemplated herein may comprise variable regions heavy chain region derived from an FcγRIIa antigen-binding fragment as described herein, and light chain region derived from a second species.

Chimeric antigen-binding fragments may be generated, for example, by genetic engineering of immunoglobulin gene segments belonging to different species.

In general, humanized, chimeric, derivative, antigen-binding fragments as contemplated herein are still capable of specifically binding to the same antigen/epitope (e.g. FcγRIIa) as the parent antibody or antigen-binding fragment from which they are derived or which they contain component/s. Typically, they may retain at least 10% of the antigen-epitope binding capacity of the parent antibody or antigen-binding fragment, or at least 25%, 50%, 60%, 70%. 80%, 90%, 95%, 99% or 100% of the antigen/epitope binding capacity of the parent antibody or antigen-binding fragment. For example, they may have a stronger binding affinity and/or binding specificity compared to the parent antibody or antigen-binding fragment.

The capacity of an antigen-binding fragment, derivative, or variant to bind specifically to an antigen/epitope that is targeted by the parent antibody or antigen-binding fragment (i.e. FcγRIIa antigen and/or epitope) can be tested using known methods in the art including, for example, competitive and non-competitive assay systems using techniques such as Western blots, radioimmunoassay, enzyme linked immunosorbent assay (ELISA), immunoprecipitation assays, “sandwich” immunoassays, immunodiffusion assays, precipitin reactions, protein A immunoassays, fluorescent immunoassays, gel diffusion precipitin reactions, complement fixation assays, immunoradiometric assays, agglutination assays and the like (see, for example, Ausubel et al., eds., Short Protocols in Molecular Biology (John Wiley & Sons, Inc., New York, 4th ed. 1999); Harlow & Lane, Using Antibodies: Laboratory Manual (Cold Spring Harbor Laboratory Press, Cold Spring Habor, N.Y., 1999).

The antigen-binding fragment derivatives may also include labelled antigen-binding fragments such as, for example, antigen-binding fragments labelled with radioactive iodine, indium, sulphur, carbon, tritium, or the like; antigen-binding fragments conjugated with avidin or biotin, antigen-binding fragments conjugated with enzymes (e.g. horseradish, glucose 6-phosphate dehydrogenase glucose oxidase, beta-D-galactosidase, alkaline phosphatase, glocoamylase, acetylcholine esterase, carboxylic acid anhydrase, malate dehydrogenase, lysozyme, or peroxidase), and antigen-binding fragments conjugated with chemiluminescent agents (e.g. acridine esters), bioluminescent agents (e.g. luciferase), or fluorescent agents (e.g. phycobiliproteins).

Processes for the preparation of antigen-binding fragments, derivatives and variants thereof, are well known, and if necessary, such known methods may be readily modified, tweaked or adapted without difficulty by persons of ordinary skill in the art in the field of immunology.

The present invention also provides nucleic acid molecules encoding the antigen-binding fragments of the invention, vectors and host cells. No particular limitation exists as to the origin of the host cell, which may be derived, for example, from bacteria, a mammal or an insect.

### Medicaments and Pharmaceutical Formulations

Medicaments and pharmaceutical formulations according to the present invention comprise antibody-binding fragments as described herein. The medicaments and pharmaceutical formulations may be prepared using methods known to those ordinary skill in the art. Non-limiting examples of suitable methods are described in Gennaro et al. (Eds), (1990), “Remington's Pharmaceutical Sciences”, Mack Publishing Co., Easton, Pennsylvania, USA.

The medicaments and pharmaceutical formulations may comprise one or more pharmaceutically acceptable carriers, excipients, diluents and/or adjuvants which do not produce adverse reaction/s when administered to a particular subject such as human or non-human animal. Pharmaceutically acceptable carriers, excipients, diluents and adjuvants are generally also compatible with other ingredients of the medicaments and pharmaceutical formulations. Non-limiting examples of suitable excipients, diluents, and carriers can be found in the “Handbook of Pharmaceutical Excipients” 4th Edition, (2003) Rowe et al. (Eds), The Pharmaceutical Press, London, American Pharmaceutical Association, Washington, and may include, for example, distilled water, saline solution, vegetable based oils such as peanut oils, safflower oil, olive oil, cottonseed oil, maize oil, sesame oil, arachis oil, or coconut oil, silicon oil, including polysiloxanes, volatile silicones, minerals oils such as lipid paraffin, soft paraffin or squalene, cellulose derivates such as methyl cellulose, ethyl cellulose, carboxymethylcellulose, sodium carboxymethylcellulose or hydroxypropylmethylcellulose, lower alkanols (for example ethanol or isopropanol), lower aralkanols, lower polyalkylene glycols or lower alkylene glycols (for example polyethylene glycol, polypropylene glycol, ethylene glycol, propylene glycol, 1,3-butylene glycol), or glycerin, fatty acid esters such as isopropyl palmitate, isopropyl myristate or ethyl oleate, polyvinylpyrridone, agar, carrageenan, gum tragacanth or gum acacia and petroleum jelly. Typically, the carrier or carriers will form from 10% to 99.9% by weight of the compositions.

In certain embodiments, medicaments and pharmaceutical formulations of the present invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, intradermal, subcutaneous, intramuscular or intravenous injection.

Solid forms of the medicaments and pharmaceutical formulations for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monosterate or glyceryl distearate. Liquid forms of the medicament and pharmaceutical formulations for oral administration may contain, in addition to the above agents, a liquid carrier. Suitable liquid carriers include water, oils, such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administrations may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

For preparation of the medicaments and pharmaceutical formulations as injectable solutions or suspensions, non-toxic parenterally acceptable diluents or carriers may be used such as Ringer's solution, isotonic saline, phosphate buffered saline, ethanol and 1,2 propylene glycol.

Emulsions for oral administration may further comprise one or more emulsifying agents. Suitable emulsifying agents include dispersing agents as exemplified above or natural gums such as guar gum, gum acacia or gum tragacanth.

Topical formulations comprise an active ingredient(s) (e.g. i.e. antibodies and/or antigen-binding fragments thereof of the present invention) together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

When formulated as drops, the medicaments and pharmaceutical formulations may comprise sterile aqueous or oily solutions or suspensions. These may be prepared by dissolving the active ingredient in an aqueous solution of bactericidal and/or fungicidal agent and/or any other suitable preservative, and optionally including a surface-active agent. The resulting solution may then be clarified by filtration, transferred to a suitable container and sterilised. For example, sterilisation may be achieved by filtration followed by transfer to a container by aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

When formulated as creams, ointments or pastes, the medicaments and pharmaceutical formulations may be semi-solid formulations of the active ingredient for external application. They may be made by mixing the active ingredient in finely divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap, a mucilage, an oil of natural origin such as almond, corn, arachis, castor or olive oil, wool fat or its derivatives, or a fatty acid such as stearic or oleic acid together with an alcohol such as propylene glycol or macrogols.

The medicaments and pharmaceutical formulations may include any suitable surfactant such as an anionic, cationic or non-ionic surfactant such as sorbitan esters or polyoxyethylene derivatives thereof. Suspending agents such as natural gums, cellulose derivatives or inorganic materials such as colloidal silicas, and other ingredients such as lanolin, may also be used.

### Methods for Treating Thrombogenic-Related Diseases

HIT is a life-threatening thrombotic complication of heparin treatment. HIT occurs when immune complexes consisting of PF4 and specific HIT immunoglobulins (IgG) are formed following sensitization of the patients by heparin. These immune complexes interact with platelets via FcγRIIa receptors, inducing receptor cross-linking, thus leading to platelet activation. The activated platelets subsequently release PF4 which amplifies the immune complex-mediated platelet activation events and also generates procoagulant microparticles which initiate the downstream coagulation pathway. The clinical consequences include venous thrombosis such as deep vein thrombosis and pulmonary embolism; arterial thrombosis such as myocardial infarction, stroke and limb gangrene which often requires amputation. The antigen-binding fragments and treatment methods herein provide an ability to block the detrimental activity of HIT antibody/PF4/polyanion immune complexes.

Vaccine-induced immune thrombotic thrombocytopenia (VITT), also known as thrombotic thrombocytopenia syndrome (TTS), is an adverse effect of COVID-19 adenoviral vector vaccines such as the ChAdOx1 nCoV-19 vaccine (AstraZeneca; University of Oxford) and the Ad26.COV2.S vaccine (Janssen; Johnson & Johnson). Although rare, VITT has been diagnosed in at least several hundred patients worldwide with a mortality rate of approximately 40%. VITT resembles HIT in that it is associated with platelet-activating antibodies against PF4 that activate cells via FcγRIIA. Anti-FcγRIIa antigen-binding fragments described herein could be used to treat VITT.

The clinical thrombotic consequences described above may also occur in “HIT-like conditions (such as viral [including COVID-19], bacterial and fungal infections) in which HIT IgG or HIT-like IgG is produced following sensitization of the patients by the infective agents. The antibody/antigen immune complexes formed can activate immune cells and platelets resulting in thrombosis and/or thrombocytopenia. In infections, the immune cells may be activated by other agonists inducing cytokine release and inflammation, which further exacerbating the thrombosis. Administration of the anti-FcγRIIa antigen-binding fragments described herein can inhibit immune cell activation and alleviate these serious conditions.

Moreover, the clinical thrombotic consequences described above may occur as a co-morbidity/mortality in diseases including but not limited to acute respiratory disease (e.g. SARS/C OVID-19), sepsis and fungal infection.

Pathogenic aspects of other immune conditions such as immune thrombocytopenia (ITP) an autoimmune bleeding disorder characterised by production of auto-reactive antibodies, drug-induced thrombocytopenia (DITP), systemic lupus erythematosus (SLE) and rheumatoid arthritis also depend on activation and/or signalling via the FcγRIIa receptor. Effective inhibition by anti-FcγRIIa antigen-binding fragments described herein could additionally alleviate these serious diseases.

ITP can be primary or secondary to other conditions. Primary ITP is defined as an isolated platelet count of less than 100×10⁹/L (reference count 150-400×10⁹/L) in the absence of other causes or conditions that may cause thrombocytopenia. Secondary ITP is due to many conditions including a number of drugs (rifampicin, vancomycin, quinine), H. pylori infection, HIV, hepatitis C, lupus, other autoimmune disorders, anti-phospholipid syndrome, and malignancy.

The identification of subjects with or at risk of developing thrombogenic-related diseases, in particular HIT and ITP, is well known to those of ordinary skill in the art. For example, in the case of HIT, the criteria for diagnosis is usually a normal platelet count before the initiation of heparin, thrombocytopenia defined as a drop in platelet count by 30% to <100×10⁹/l or a drop of >50% from the subject's baseline platelet count, the onset of thrombocytopenia typically 5-10 days after initiation of heparin treatment, which can occur earlier with previous exposure to heparin (within 100 days), haematology, acute thrombotic event and HIT antibody seroconversion.

For example, primary ITP is usually defined by low platelet count, normal bone marrow and the absence of other causes of thrombocytopenia. ITP can be diagnosed using standard tests including: urinalysis, CBC with differential, haematology, coagulation, serum chemistry, surfactant D, erythrocyte sedimentation rate, and C-reactive proteins. HIT and ITP (primary or secondary) may develop bleeding, thrombosis and end-organ damage.

Once a subject has been determined as suffering or being at risk of developing a thrombogenic-related disease, such as HIT and ITP, the methods of the present invention may be used for preventing or treating those diseases in the subject.

It will be understood that “treating” within the context of the methods described herein refers to the alleviation, in whole or in part of symptoms associated with either ITP or HIT, or slowing, inhibiting or halting further progression of either ITP or HIT. Typically, a therapeutic response can be considered as an increase in platelet count above those thresholds that place the subject at risk for bleeding (30×10⁹), improvement/resolution of thrombosis damage and end-organ damage.

A non-limiting example of the methods of the present invention includes administration of the antigen-binding fragment/s of the invention to a subject with moderate to severe ITP (primary or secondary) wherein the subject has either bleeding, thrombosis, deep vein thrombosis (DVT), petechiae or bruising. A further non-limiting example of the present invention includes administration of antigen-binding fragment, to a subject with moderate to severe HIT wherein the subject has either had a reduced or inadequate response to warfarin or other anticoagulants.

A non-limiting example of the present invention includes administration of the antigen-binding fragment to a subject with an immune condition with thrombocytopenia and/or thrombosis and/or end-organ damage. Further non-limiting examples include treatment of NETs-induced thrombo-embolism, organ-injury, other NETs-associated disorders, ITP associated with drugs, viral and bacterial infections or antibody treatments, antiphospholipid syndrome, cancer-induced thrombocytopenia and thrombo-embolism, autoimmune or inflammatory diseases involving FcγRII (CD32) and involving either one or more of the following cells; platelets, neutrophils, monocytes, macrophages, eosinophils, basophils and mast cells. Some embodiments of the invention involve the administration of the antigen-binding fragment to a subject with a thrombogenic-related disease which involves the binding of immune complexes to FcγRIIA.

In a particular embodiment of the invention, the method comprises the administration of a combination of one or more antibody-binding fragments, wherein the combination comprises administering to the subject, two, three, four, five, six, or more antibody-binding fragments described herein.

In a further embodiment, the method comprises administering to a subject two or more treatments that act together to inhibit FcγRIIa binding.

In some embodiments the antigen-binding fragment/s are administered to a subject in an amount and time that is sufficient to generate an improvement, preferably a sustained improvement, in at least one indicator of disease severity that can be treated. Various indicators may be used to assess whether the amount and time of treatment is sufficient. Such indicators include, for example, clinically recognised indicators of disease severity, symptoms and/or manifestations of the disorder or condition. The degree of improvement is generally determined by a physician, who may make the determination using signs, symptoms, biopsies or other test results.

The subject may be any animal that can benefit from the administration of antigen-binding fragment. In some embodiments, the subject is a mammal, for example, a human, a dog, a cat, a horse, a cow, a pig, a primate, or rodent (e.g. a mouse or rat).

The methods may involve the administration of a “therapeutically effective amount” of antigen-binding fragment according to the invention. A “therapeutically effective amount” will be understood to refer to an amount of antigen-binding fragment that alleviates, in whole or in part symptoms associated with either HIT or ITP, or slows, inhibits or halts further progression or worsening of those symptoms, in a subject with or at risk of developing either HIT or ITP.

The therapeutically effective amount may vary depending upon the route of administration, the particular antigen-binding fragment and the dosage form. Effective amounts of antigen-binding fragments, according to the present invention typically fall in the range of about 0.001 up to 100 mg/kg/day, for example in the range of about 0.05 up to 20 mg/kg/day. Typically, the antigen-binding fragments provide a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

Generally, an effective dosage of antigen-binding fragment, according to the present invention is expected to be in the range of about 0.0001 mg to about 1000 mg of active component/s (i.e. of antigen-binding fragment according to the present invention) per kg of body weight per 24 hours; typically, about 0.001 mg to about 750 mg per kg body weight per 24 hours; about 0.01 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m² of active component/s (i.e. antigen-binding fragments according to the present invention). Generally, an effective dosage is expected to be in the range of about 0.1 to about 500 mg/m², about 1 to about 250 mg/m², about 1 to about 200 mg/m², about 1 to about 150 mg/m², about 1 to about 100 mg/m², about 1 to about 50 mg/m², about 1 to about 25 mg/m², or about 1 to about 5 mg/m².

Typically, for therapeutic applications, the treatment would be for the duration of either conditions ITP or HIT in the subject. Further, it will be apparent to one of ordinary skill in the art that the optimal quantity and spacing of individual dosages will be determined by the nature and the severity of either ITP or HIT, the form, route and site of administration, and the nature of the particular individual being treated. Also, such optimal conditions can be determined by conventional techniques.

In many instances, it will be desirable to have several or multiple administrations of the antigen-binding fragments. In certain embodiments, a given dosage may be administered 1, 2, 3, 4, 5, 6, 7, 8, 9, 10, or more times. The dosage may be administered once to the subject, or more than once at certain interval/s, for example, once a day, once a week, twice a week, three times a week, once a month, twice a month, three times a month, once every two months, once every three months, once every six months, or once a year. The duration of treatment and any changes to the dosage and/or frequency of treatment can be altered or varied during the course of treatment in order to meet the particular needs of the subject. It will be apparent to one of ordinary skill in the art that the optimal course of treatment can be ascertained using conventional course of treatment determination tests.

In certain embodiments, the antigen-binding fragments can be formulated for various routes of administration, for example, by parenteral (e.g. intradermal, intravenous, intraspinal, intraperitoneal, subcutaneous or intramuscular), or by way of an implanted reservoir. Systemic or parenteral administration includes, but is not limited to, intraperitoneal, intramuscular, subcutaneous, intramucosal, and intravenous injections. In one embodiment, the antigen-binding fragments are administered by an intravenous route. Non-limiting examples of acceptable routes of mucosal administration including intranasal, buccal, genital tract, rectal, intratracheal, skin and the gastrointestinal tract.

In certain methods described herein, antigen-binding fragments may be administrated in combination with other agents or therapies, including administration in combination with current standard of care with anticoagulants or alternative anticoagulants. The antigen-binding fragments may be components of pharmaceutical formulations or medicaments as are described herein. Non-limiting examples of additional agents or therapies for HIT include; argatroban, bivalirudin, fondaparinux, warfarin, danaparoid, rivaroxaban, dabigatran and apixaban. Non-limiting examples of additional agents or therapies for ITP include prednisone, gamma globulin, anti-Rho(D) immune globulin (WinRho), rituximab, danazol, azathioprine, cyclophosphamide, vincristine, vinblastine and romiplostim. The antigen-binding fragments may be administered to the subject simultaneously with the additional agents or therapies. Such additional agent/s may be administered orally or by another route, for example via IV injection. Additionally, or alternatively, the antigen-binding fragments thereof may be administered to the subject before or after the additional agent/s or therapy/ies are administered.

### EXAMPLES

The present invention will now be described with reference to specific Examples, which should not be construed as in any way limiting.

### Example One

Overview

The variable regions of IV.3, a mouse monoclonal antibody (MoAb) specific for the FcγRIIA receptor, were cloned by extracting RNA from IV.3-expressing hybridoma cells. The variable domains of the heavy and light chain were humanized and joined with a 20 amino-acid peptide linker, GGGGWAWVWLTETAVGGGGS. The linker contains a sequence (WAWVWLTETAVA) that also recognises FcγRIIA.

Description of the Molecules Created:

HRU5: Humanized variable heavy and variable light chains derived from the IV.3 MoAb linked with a GGGGWAWVWLTETAVGGGGS linker.

HRU6: HRU5 linked to lepirudin (an anticoagulant peptide) by a Ser₂Gly₁₂ linker.

HRU7: HRU5 linked to bivalirudin (an anticoagulant peptide) by a Ser₂Gly₁₂ linker.

The constructs were expressed in E. coli and purified by affinity chromatography. Binding to human platelets in vitro and functional activity in vitro and in vivo were characterized.

Materials and Methods

(i) Patients

Samples were selected randomly from stored sera collected with informed consent from patients with HIT. The diagnosis of HIT was made according to the criteria outlined in Chong and Isaacs, “Heparin-induced thrombocytopenia: What clinicians need to know” Thromb Haemost. 2009; 101(2):279-283. The study was approved by the South Eastern Sydney Illawarra/Eastern Sydney Area Health Service Ethics Committees. Blood and plasma from healthy donors were obtained with informed consent.

(ii) Molecular Cloning and Humanization of the scFv

Total RNA was isolated from IV.3 hybridoma cells with the RNeasy Mini Kit (Qiagen, Germany) according to the manufacturer's instructions. The first-strand cDNA was synthesized with the SuperScript™ III First-Strand Synthesis SuperMix for qRT-PCR (Invitrogen, Carlsbad, CA). PCR amplification of the heavy chain and light chain variable regions was conducted using specific primers as follows:

Heavy Chain Forward Primer
5′GGCCAGTGGATAGTCAGATGGGGGTGTCGTTTTGGC-3′
and
Reverse Primer
5′-GAGGTGAAGCTGGTGGAGTC-3′.
Light Chain Forward Primer
5′-GGATACAGTTGGTGCAGCATC-3′
and
Reverse Primer
5′-GATATTGTGATGACGCAGGCT-3′.

For VH, PCR was performed at 95° C. for 5 min, 35 cycles at 94° C. for 45 sec, 50° C. for 72° C. for 90 sec, followed by 72° C. for 5 min; for VL, the conditions were 95° C. for 5 min, cycles at 94° C. for 45 sec, 54° C. for 45 sec, 72° C. for 90 sec, followed by 72° C. for 5 min. Both amplified fragments were cloned into pCR®-Blunt vector (Invitrogen). The nucleotide sequences of both variable fragments were confirmed with BigDye Terminator v3.1 Sequencing Reagent (Applied Biosystems, CA). The scFv was humanized using the Complementarity Determining Region (CDR)-grafting and point mutation approach. The sequences of the six CDRs were preserved and selected amino acid residues were changed in the framework regions (FRs) to reflect the subgroups of human VH and VL domains. This procedure was conducted with reference to the IMGT database available at http://www.biochem.unizh.ch/antibody. The sequence encoding the humanized VH and VL domains, fused with a short linker Glyu-Sers (Sequence 9) was chemically synthesized (DNA2.0, Menlo Park, CA) with codon optimization for increased protein expression in bacteria (Sequence 3). The constructs were cloned into the pET11a expression vector and included HIS6-tags at the C-terminus for purification. The vector was transformed into OverExpress™ CD41(DE3) competent cells (Lucigen Corporation, Middleton, WI) already containing the pKJE7 chaperone plasmid (Takara Bio Inc, Otsu, Japan). Transformed cells were grown in the presence of ampicillin (100 μg/ml) and chloramphenicol (20 μg/ml). The DNA of HRU5, HRU6 and HRU7 was synthesized using GenScript and the protein was expressed in OverExpress™ CD41(DE3) E. coli cells.

(iii) Protein Expression and Purification

Expression of scFvs was induced with 0.05% L-arabinose and 0.5 mM IPTG for 4 h at ° C. Bacterial cells were lysed in lysis buffer (50 mM Tris-Cl, 300 mM NaCl, 0.5% Triton X-100, 2 mM phenylmethanesulfonylfluoride, EDTA-free protease inhibitors (Roche Diagnostics, Castle Hill, Australia)), purified by HIS affinity chromatography and identity confirmed by Western blot with an anti-Penta-HIS antibody. The purified scFv was examined with SDS-PAGE under reducing conditions and detected by Western Blotting with Penta-HIS antibody (Qiagen; 34660) followed by incubation with polyclonal rabbit anti-mouse immunoglobulins conjugated with horseradish peroxidase. The signal was developed with Western Lightning® Plus-ECL, Enhanced Chemiluminescence Substrate (Perkin-Elmer, Waltham, MA, USA) and detected with ImageQuant LAS4000 imager (GE Healthcare, Uppsala, Sweden).

(iv) Platelet Binding for Flow Cytometry Analysis

Platelet-rich plasma (PRP) was obtained from whole blood after centrifugation at 200×g for 10 min. Washing/Suspension buffer (PBS, 0.5% BSA, 25 mM EDTA, pH 6.8) was used for platelet preparation throughout the experiment. 4 μl of PRP was incubated with various concentrations of fluorescently labelled HRU molecules for 30 min. Cells were washed once with 500 μl of buffer (PBS, 0.5% BSA, 25 mM EDTA, pH 6.8), suspended in 200 μl of PBS and analysed by flow cytometry (Cantoll, BD Biosciences).

(v) Platelet Aggregation

Platelet aggregation assays were performed by mixing samples at 1200 rpm, 37° C. for up to 25 min in an aggregometer (Chrono-log Aggro/Link™). The reaction mixture (500 μl) consisted of 300 μl of healthy donor PRP, HIT patient serum or purified Total HIT IgG (up to 14 μM, adjusted according to different donor platelets), heparin (0.5 IU/ml) and various concentrations of HRU5-HRU7. The degree of aggregation was determined by the increase in light transmittance. Platelet aggregation levels over 20% were considered positive.

For thrombin induced platelet aggregation samples were mixed as described above in the presence of 0.3 U of thrombin.

(vi) Serotonin Release Assay (SRA)

SRA is the gold standard to confirm the presence of platelet-activating HIT antibodies. Platelets from a healthy donor were labelled with Hydroxytryptamine Binoxalate, 5-[2-¹⁴C]-(Serotonin) (¹⁴C-5HT) (1.5 μl/ml) and mixed with heparin (0.1 (low dose) or 100 IU/ml (high dose)) in the presence of HIT serum. The protein of interest was then added to the reaction and incubated at room temperature for 1 h. After centrifugation the supernatant was collected and the ¹⁴C-serotonin released was measured in a scintillation counter. The percentage of ¹⁴C-serotonin released was calculated by determining the proportion in the supernatant relative to the remaining ¹⁴C-serotonin in platelets. A test result is defined as positive if more than 20% serotonin release is detected with a therapeutic heparin concentration of (0.1 IU/ml) but not with a high dose of heparin (100 IU/ml).

(vii) Microfluidics Device and Image Acquisition

Vena8 Fluoro+™ biochip micro-channels were coated with von Willebrand factor (vWf) at a concentration of 200 μg/ml at 4° C. overnight. After washing with PBS, the micro-channels were blocked with PBS/1% BSA for 30 min. Whole blood anti-coagulated with ACD was labelled with antibodies to detect either platelets and neutrophils or Sytox green to detect extracellular DNA and incubated for 10 min at RT in the dark.

The assay was performed at 37° C. in a Venaflux micro-fluidics device (Cellix Ltd. Dublin, Ireland) in the absence or presence of HRU molecules with patient HIT IgG or normal IgG as control at a fluid shear rate of 20 dyne/cm² for up to 460 sec. Flow chambers were mounted on a fluorescent microscope (Zeiss Axio Observer.A1) and fluorescence images from different microscopic fields were captured in real time with a Q-Imaging EXi Blue™ camera (Qlmaging, Surry, BC, Canada) driven by Venaflux software (Cellix Ltd. Dublin, Ireland). The fluorescence images were analysed with Image-Pro Premier 9.1 software (Media Cybernetics, Inc, Rockville, MD, USA). Deposition of thrombus component (platelets, neutrophils and DNA) was measured by calculating area coverage.

(viii) Thrombin Time Assay

The STA-Thrombin kit (Diagnostica STAGO S.A.A.) was used for the determination of the thrombin time by a haemostasis analyser. Plasma was prepared from ACD anticoagulated normal blood by centrifugation for 10 min at 2500×g. 500 μl of undiluted plasma, in the absence and presence of different inhibitor concentrations (e.g., HRU6 and HRU7), was analysed. Thrombin time of the plasma was determined by the analyser.

(ix) Animal Model of HIT Using FcγRIIA/hPF4 Double Transgenic Mice

The mice used in these experiments were described in Reilly et al., “Heparin-induced thrombocytopenia/thrombosis in a transgenic mouse model requires human platelet factor 4 and platelet activation through FcγRIIA” Blood. 2001; 98(8):2442-2447. Animals were injected intravenously (iv) with HIT-like MoAb KKO. Heparin was injected intraperitoneally at 1 U/g following IgG injection. In some experiments, HRU molecules were also injected via the iv route. For in vivo platelet labelling anti-CD42c-Dylight649 (Emfret) was injected iv at 1 μg/g. Platelet counts were measured before treatment and at 1 h, 3 h and 5 h following treatment. Lungs were harvested 4 h after treatment, fixed in formalin and scanned for fluorescence in an IVIS Lumina Spectrum CT Spectrometer (PerkinElmer).

Results

Sequences

The sequences of the murine scFv as well as the humanized molecules (HRU5 to HRU7) and linkers are shown in Table 1 as SEQ ID NOs 1-16. The scFvs are arranged VH-linker-VL.

TABLE 1
Sequence information

| SEQ | | |
|---|---|---|
| ID | | |
| NO: | DESCRIPTION | SEQUENCE |
| 1 | scFv murine protein | VH: |
| | sequence derived | EVKLVESGPE LKKPGETVKI SCKASGYTFT NYGMNWVKQA |
| | from IV.3 hybridoma | PGKGLKWMGW LNTYTGESIY PDDFKGRFAF SSETSASTAY |
| | cells | LQINNLKNED MATYCARGD YGYDDPLDYW GQGTSVTVSS |
| | CDRs are underlined | VL: |
| | | DIVMTQAAPS VPVTPGESVS ISCRSSKSLL HTNGNTYLHW |
| | | FLQRPGQSPQ LLIYRMSVLA SGVPDRESGS GSGTAFTLSI |
| | | SRVEAEDVGV FYC MQHLEYP LTFGAGTKLE LKRA |
| 2 | Humanized scFv | EVQLVESGPE LKKPGETVKI SCKASGYTFT NYGMNWVKQA |
| | Changes from murine | PGKGLKWMGW LNTYTGESIY PDDFKGRFAF SLETSASTAY |
| | to human-like | LQINNLKSED TATYFCARGD YGYDDPLDYW GQGTSVTVSS |
| | sequences are in bold. | GGGGSGGGGS GGGGSDIVMT QAPPSVPVTP GESVSISCRS |
| | This scFv is referred | SKSLLHTNGN TYLHWFLQKP GQSPRLLIYR MSVLASGVPD |
| | to herein as the | RFSGSGSGTD FTLKISRVEA EDVGVYYCMQ HLEYPLTFGA |
| | parental scFv. CDRs | GTKLEIKRAH HHHHH |
| | are underlined. The | |
| | linker is in italics. | |
| 3 | HRU5 | EVQLVESGPE LKKPGETVKI SCKASGYTFT NYGMNWVKQA |
| | The CDRs are | PGKGLKWMGW LNTYTGESIY PDDFKGRFAF SLETSASTAY |
| | underlined. The linker | LQINNLKSED TATYFCARGD YGYDDPLDYW GQGTSVTVSS |
| | is in italics. | GGGG [symbol] [symbol] GGGGS DIVMTQAPPS VPVTPGESVS |
| | | ISCRSSKSLL HTNGNTYLHW FLQKPGQSPR LLIYRMSVLA |
| | | SGVPDRFSGS GSGTDFTLKI SRVEAEDVGV YYCMQHLEYP |
| | | LTFGAGTKLE IKRAHHHHHH |
| 4 | CDR1 of HRU5 (VH) | NYGMN |
| 5 | CDR2 of HRU5 (VH) | WLNTYTGESIYPDDFKG |
| 6 | CDR3 of HRU5 (VH) | GDYGYDDPLDY |
| 7 | CDR1 of HRU5 (VH) | RSSKSLLHTNGNTYLH |
| 8 | CDR2 of HRU5 (VH) | RMSVLAS |
| 9 | CDR3 of HRU5 (VH) | MQHLEYPLT |
| 10 | Linker sequence of | GGGGWAWVWLTETAVGGGGS |
| | HRU5 | |
| 11 | FcγRIIA binding | WAWVWLTETAV |
| | peptide | |
| 12 | HRU6 ((HRU5 linked | EVQLVESGPE LKKPGETVKI SCKASGYTFT NYGMNWVKQA |
| | to lepirudin) | PGKGLKWMGW LNTYTGESIY PDDFKGRFAF SLETSASTAY |
| | Lepirudin sequence in | LQINNLKSED TATYFCARGD YGYDDPLDYW GQGTSVTVSS |
| | bold font | GGGGWAWVWL TETAVGGGGS DIVMTQAPPS VPVTPGESVS |
| | Linkers are in italics | ISCRSSKSLL HTNGNTYLHW FLQKPGQSPR LLIYRMSVLA |
| | | SGVPDRFSGS GSGTDFTLKI SRVEAEDVGV YYCMQHLEYP |
| | | LTFGAGTKLE IKRAGGGGSG GGGSGGGGLT YTDCTESGQN |
| | | LCLCEGSNVC GQGNKCILGS DGEKNQCVTG EGTPKPQSHN |
| | | DGDFEEIPEE YLQ |
| 13 | Linker sequence | GGGGSGGGGSGGGG |
| | between scFv and | |
| | lepirudin | |
| 14 | HRU7 ((HRU5 linked | EVQLVESGPE LKKPGETVKI SCKASGYTFT NYGMNWVKQA |
| | to bivalirudin) | PGKGLKWMGW LNTYTGESIY PDDFKGRFAF SLETSASTAY |
| | Bivalirudin sequence | LQINNLKSED TATYFCARGD YGYDDPLDYW GQGTSVTVSS |
| | bold font in | GGGGWAWVWL TETAVGGGGS DIVMTQAPPS VPVTPGESVS |
| | Linkers are in italics | ISCRSSKSLL HTNGNTYLHW FLQKPGQSPR LLIYRMSVLA |
| | | SGVPDRFSGS GSGTDFTLKI SRVEAEDVGV YYCMQHLEYP |
| | | LTFGAGTKLE IKRAGGGGSG GGGSGGGGFP RPGGGGNGDF |
| | | EEIPEEYL |
| 15 | HRU5 VH | EVQLVESGPE LKKPGETVKI SCKASGYTFT NYGMNWVKQA |
| | | PGKGLKWMGW LNTYTGESIY PDDFKGRFAF SLETSASTAY |
| | | LQINNLKSED TATYFCARGD YGYDDPLDYW GQGTSVTV |
| 16 | HRU5 VL | DIVMTQAPPS VPVTPGESVS ISCRSSKSLL HTNGNTYLHW |
| | | FLQKPGQSPR LLIYRMSVLA SGVPDRFSGS GSGTDFTLKI |
| | | SRVEAEDVGV YYCMQHLEYP LTFGAGTKLE IKRA |
| 17 | Longer version of | MKSLITPITA GLLLALSQPL LAEVQLVESG PELKKPGETV |
| | HRU5 | KISCKASGYT FTNYGMNWVK QAPGKGLKWM GWLNTYTGES |
| | | TYPDDFKGRF AFSLETSAST AYLQINNLKS EDTATYFCAR |
| | | GDYGYDDPLD YWGQGTSVTV SSGGGGWAWV WLTETAVGGG |
| | | GSDIVMTQAP PSVPVTPGES VSISCRSSKS LLHTNGNTYL |
| | | HWFLQKPGQS PRLLIYRMSV LASGVPDRES GSGSGTDFTL |
| | | KISRVEAEDV GVYYCMQHLE YPLTFGAGTK LEIKRAEQKL |
| | | ISEEDLHHHH HH |
| 18 | Longer version of | KSLITPITAGLLLALSQPLLAEVQLVESGPELKKPGETVKISCKASGYT |
| | HRU6 | FTNYGMNWVKQAPGKGLKWMGWLNTYTGESIYPDDFKGRFAFSLE |
| | | TSASTAYLQINNLKSEDTATYFCARGDYGYDDPLDYWGQGTSVTVS |
| | | SGGGGWAWVWLTETAVGGGGSDIVMTQAPPSVPVTPGESVSISCRSS |
| | | KSLLHTNGNTYLHWFLQKPGQSPRLLIYRMSVLASGVPDRFSGSGSG |
| | | TDFTLKISRVEAEDVGVYYCMQHLEYPLTFGAGTKLEIKRAGGGGSG |
| | | GGGSGGGGLTYTDCTESGQNLCLCEGSNVCGQGNKCILGSDGEKNQ |
| | | CVTGEGTPKPQSHNDGDFEEIPEEYLQHHHHHH |
| 19 | Longer version of | KSLITPITAGLLLALSQPLLAEVQLVESGPELKKPGETVKISCKASGYT |
| | HRU7 | FTNYGMNWVKQAPGKGLKWMGWLNTYTGESIYPDDFKGRFAFSLE |
| | | TSASTAYLQINNLKSEDTATYFCARGDYGYDDPLDYWGQGTSVTVS |
| | | SGGGGWAWVWLTETAVGGGGSDIVMTQAPPSVPVTPGESVSISCRSS |
| | | KSLLHTNGNTYLHWFLQKPGQSPRLLIYRMSVLASGVPDRFSGSGSG |
| | | TDFTLKISRVEAEDVGVYYCMQHLEYPLTFGAGTKLEIKRAGGGGSG |
| | | GGGSGGGGFPRPGGGGNGDFEEIPEEYLHHHHHH |

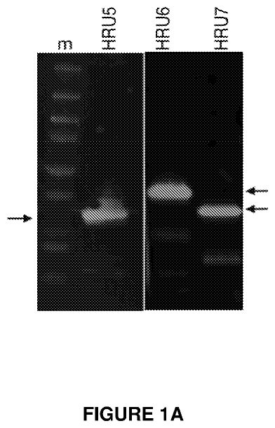

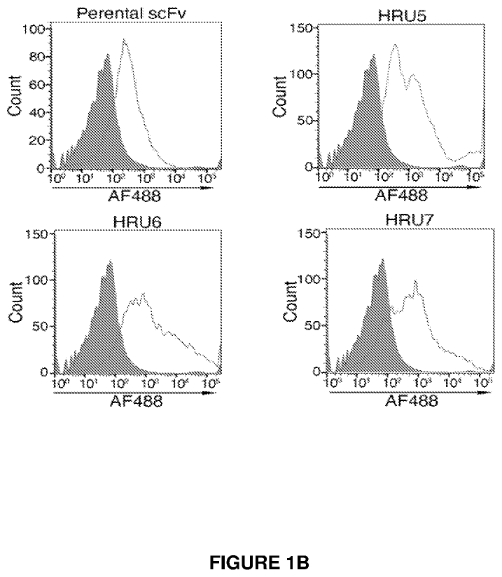

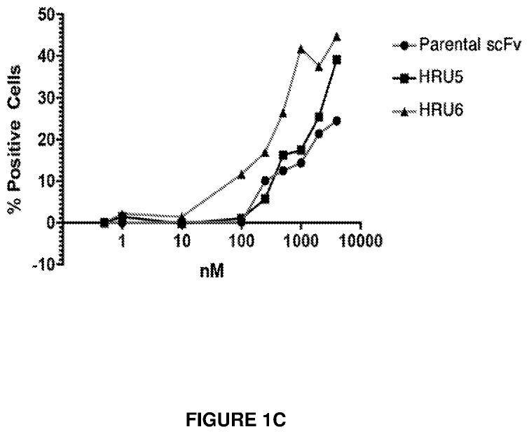

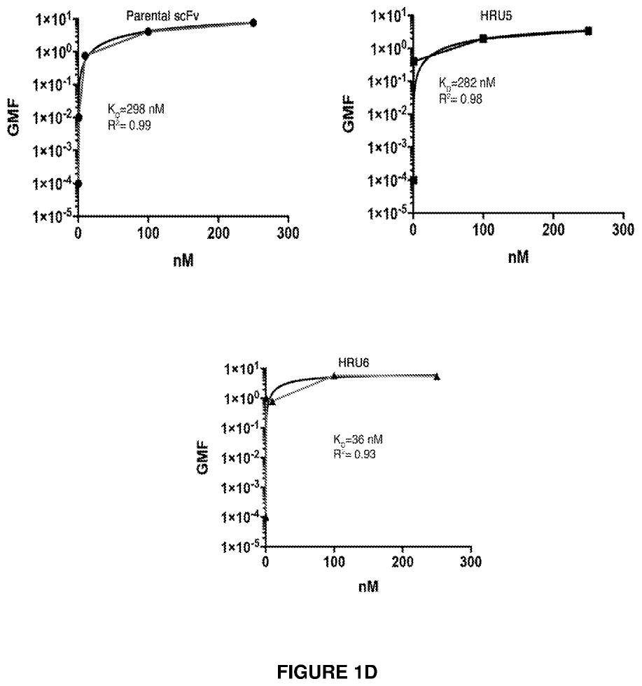

The purified proteins obtained from bacterial expression are shown in FIG. 1A. Based on protein sequence, the predicted molecular weights (in kDa) are HRU5: 28.5; HRU6: 35.5; HRU7: 30.7.

HRU5 to HRU7. Enhanced Binding to Human Platelets

The capacity of HRU molecules to interact with human platelets is shown in FIG. 1B. These experiments indicate that all scFvs (the parental scFv and HRU5-HRU7) demonstrate strong binding to human platelets. HRU5-7, particularly HRU6 shows unexpectedly greater binding than the parental scFv. Percentage binding of various concentrations of parental scFv, HRU5 and HRU6 are shown in FIG. 1C.

The presence of the functional linker (SEQ ID NOs 10 and 11) led to an increase in binding affinity in HRU5 relative to the parental scFv (K_{D} 298 nM vs 282 nM) (FIG. 1D). Addition of the anticoagulant moiety lepirudin (HRU6) led to a surprising 8-fold increase in binding affinity (HRU6 K_{D} 36 nM) (FIG. 1D). This increase in binding by HRU6 is not an additive effect of the functional linker, since the K_{D} of the linker alone is 500 nM. It was unexpected that using a weak binding peptide as a linker (HRU5) or adding an anticoagulant peptide (HRU6) would both lead to such a large increase in binding.

Effective Inhibition of Platelet Aggregation

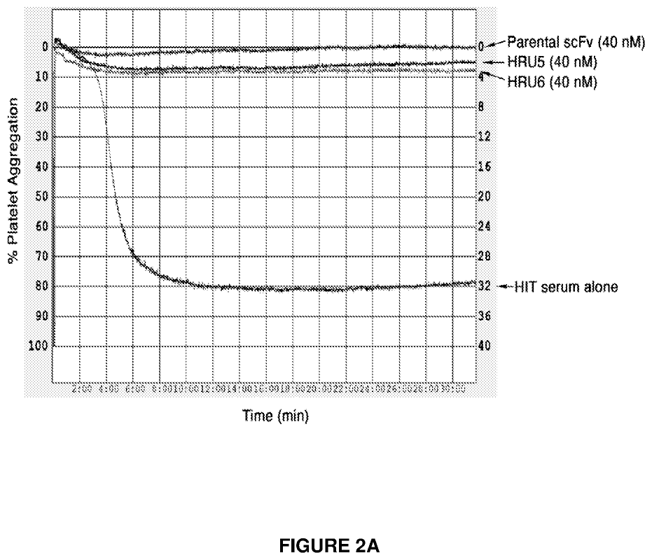

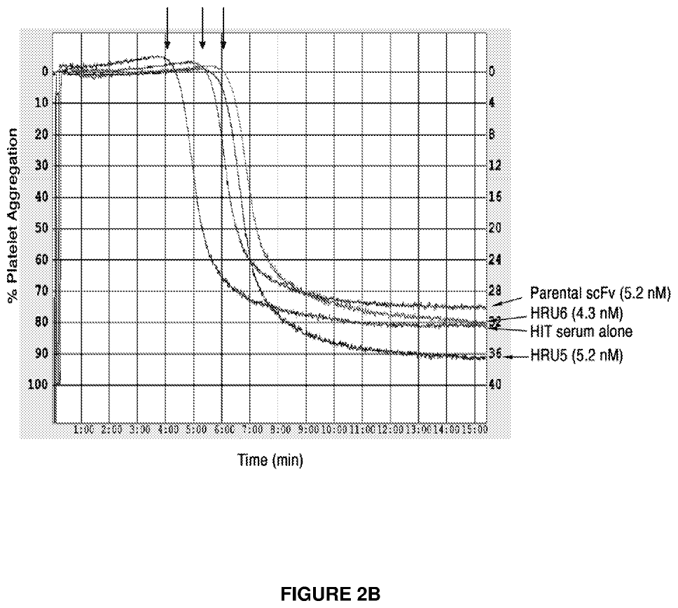

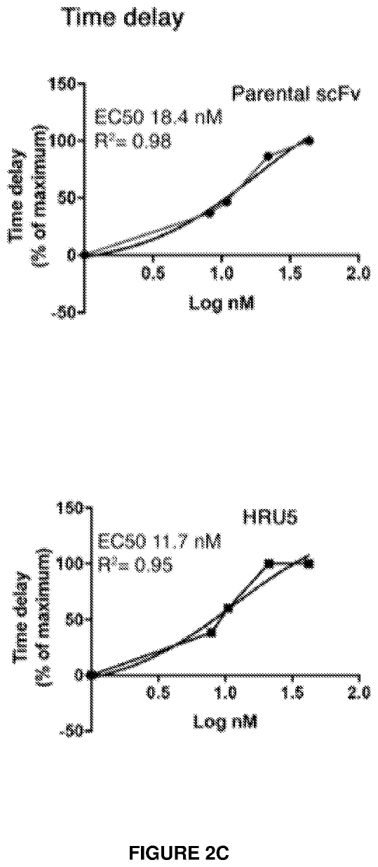

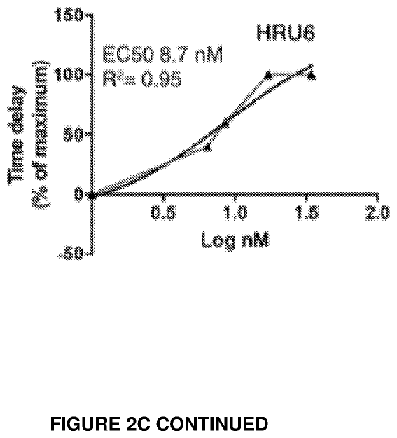

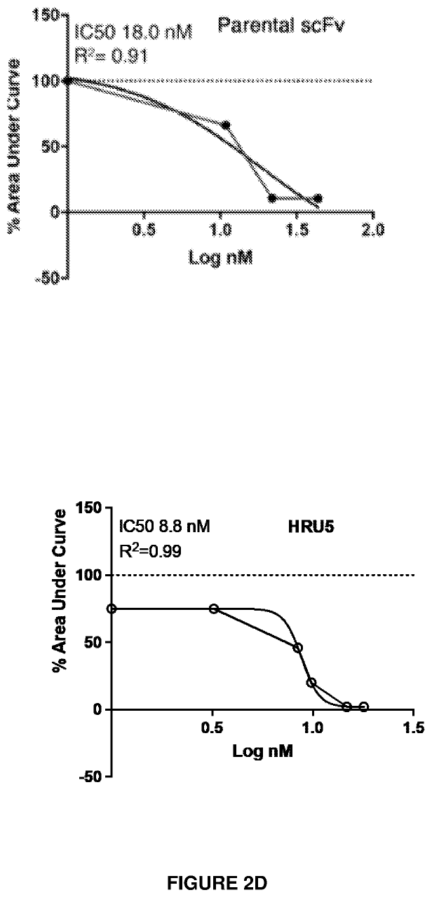

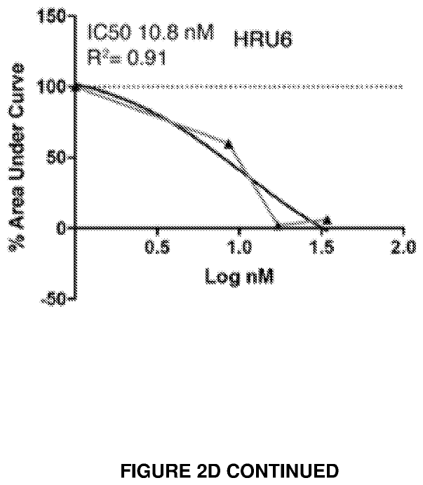

To ascertain whether the scFvs were capable of inhibiting HIT antibody-induced platelet aggregation in vitro, parental scFv, HRU5 or HRU6 were added to reactions consisting of human platelets, HIT serum and heparin. In the absence of HRU molecules, HIT serum induced strong platelet aggregation (FIG. 2A). HRU scFvs completely inhibited HIT serum induced aggregation at nanomolar concentrations (40 nM). These results show that the scFvs are functional molecules able to block platelet aggregation induced by HIT immune complexes.

Determination of 50% Effective and Inhibitory Concentrations

Measurement of 50% effective concentration (EC50) was carried out measuring time delay (i.e., increase in time elapsed before platelets start aggregating). With antibody/IC-induced platelet aggregation, time delay (time lapse) is a more sensitive measure of platelet aggregability compared with the extent of platelet aggregation as different antibodies invariably cause the same maximum extent of platelet aggregation after variable time delays or time lapses (as illustrated in FIG. 2B). Various concentrations of parental scFv, HRU5 or HRU6 were used. Plots were fitted to non-linear regression analysis (sigmoidal dose-response curves) (FIG. 2C). Similarly, non-linear regression analysis (sigmoidal dose-response curves) was used to determine the 50% inhibitory concentration (IC50). Here, the degree of inhibition of platelet aggregation was determined at various concentrations and analysed as shown in FIG. 2D. These data demonstrate the increase in activity (lower EC50 and IC50) for HRU5 and HRU6 relative to the parental scFv.

Overall, the above results show greater inhibitory activities of HRU when compared to the parental scFv, HRU6>HRU5>parental scFv.

Anti-Thrombus Activity in Whole Blood

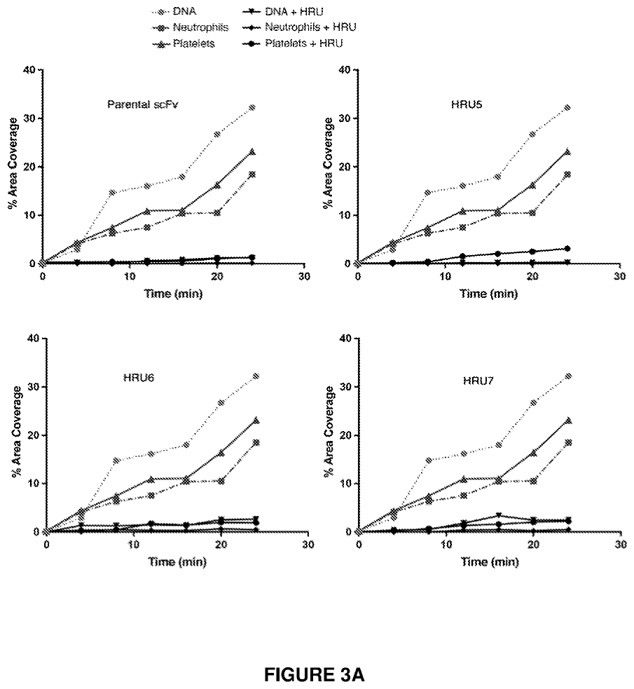

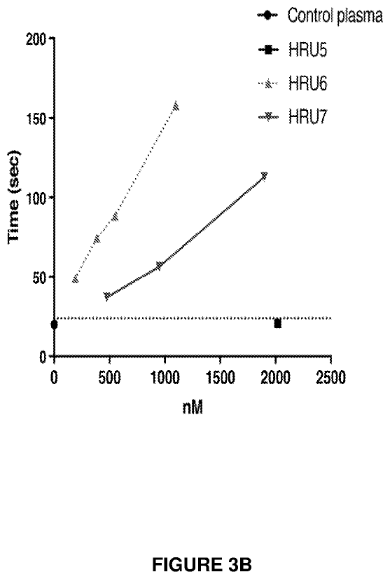

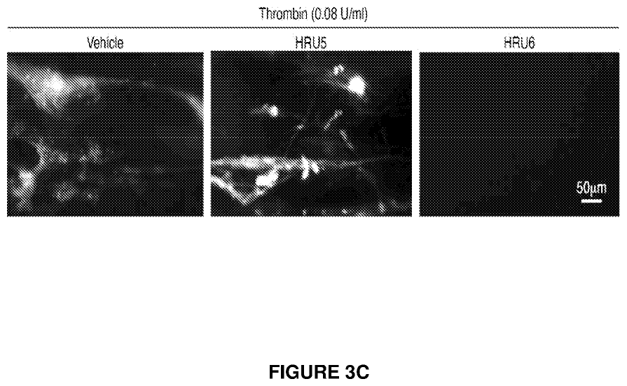

Thrombus formation in the circulation occurs in the context of flow-dependent contact between platelets, white cells, plasma and the vessel wall. Microfluidics devices allow quantitative evaluation of the activity of potential anti-thrombotic agents in a system that closely imitates in vivo conditions. The HIT condition was reconstituted using whole blood from non-medicated normal donors anti-coagulated with EDTA on a Vena8 Fluoro+™ biochip coated with vWf at a shear rate of 20 dyne/cm² at 37° C. Apart from platelets, thrombi also consist of extracellular DNA and neutrophils. Complete inhibition of deposition of platelets, DNA and neutrophils was observed in the presence of HRU5-7. This is shown in FIG. 3A. These data indicate that HUR5-7 are effective inhibitors of HIT immune complex-induced thrombus formation under flow conditions.

Anti-Thrombin Activity of HRU6 and HRU7

HRU6 and HRU7 also contain anti-thrombin activity provided by the lepirudin and bivalirudin moieties respectively. The Hemoclot thrombin inhibitor assay was used to determine the anti-clotting activity. The normal clotting time range is 15-24 sec. Increasing concentrations of HRU6 and HRU7 resulted in significant increases in thrombin clotting time (FIG. 3B). HRU5, which does not possess anti-thrombin activity, did not influence clotting time. Both HRU6 and HRU7 inhibit thrombin-induced platelet aggregation in a dose dependent manner. FIG. 3C shows that HRU6 inhibits completely fibrin deposition induced by the addition of thrombin to whole blood in a microfluidics chamber.

Inhibition of Platelet Activation

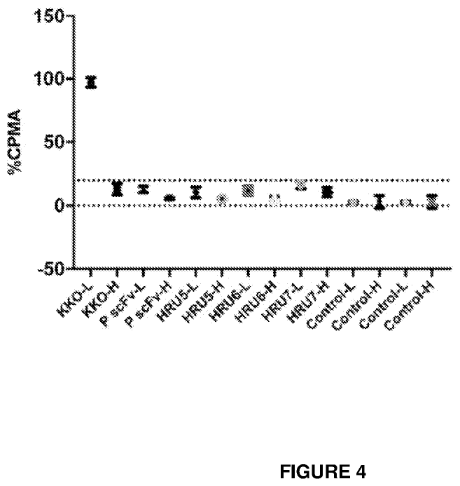

The serotonin release assay used to measure platelet activation remains widely used as a functional assay for the detection of the HIT antibodies. As shown in FIG. 4, in the absence of HRUs, HIT serum and low dose heparin (0.1 IU/ml) caused strong ¹⁴C-serotonin release while levels of ¹⁴C-serotonin released at high heparin concentrations were not significant (100 IU/ml) (high heparin concentrations dissociate the HIT immune complex and are used as a control in these assays). When HRUs were added, however, a potent reduction in the amount of ¹⁴C-serotonin released at 0.1 IU/ml heparin was observed, indicating that these molecules are potent inhibitors of HIT immune complex-induced platelet activation.

Activity of HRUs in an Animal Model of HIT Using FcγRIIA/hPF4 Double Transgenic Mice

Reconstitution of the HIT condition requires expression of both human FcγRIIA and human PF4. In this experiment, a double transgenic mouse line (Tg mice) expressing both human proteins was used. These animals have previously been established as a model of HIT.

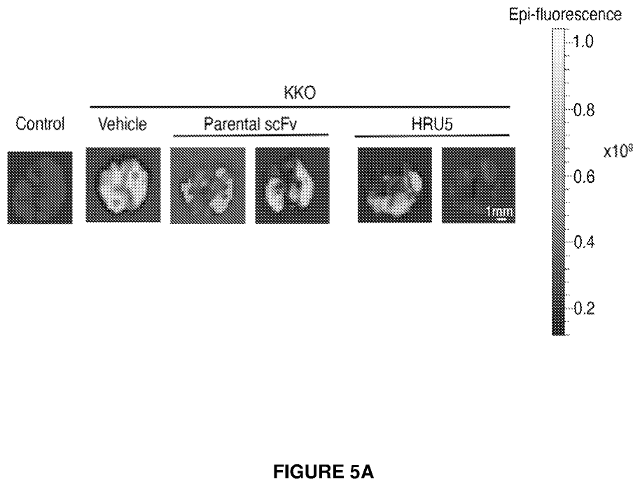

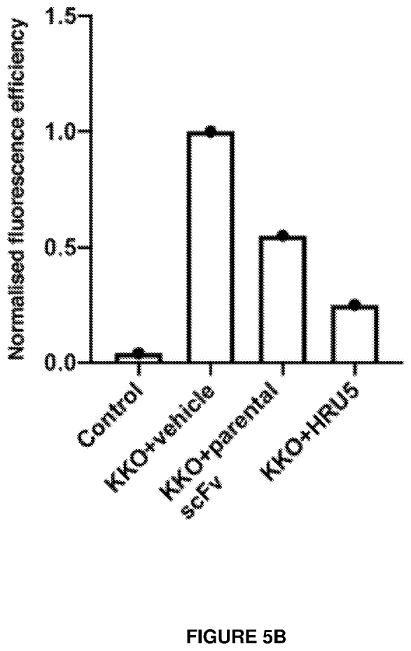

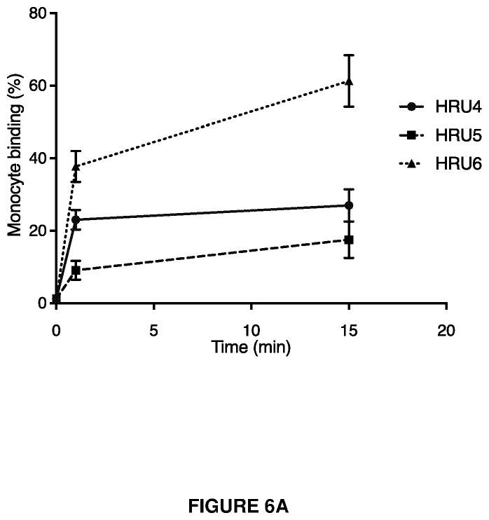

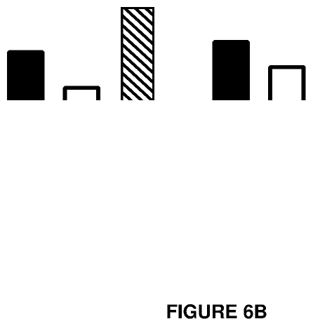

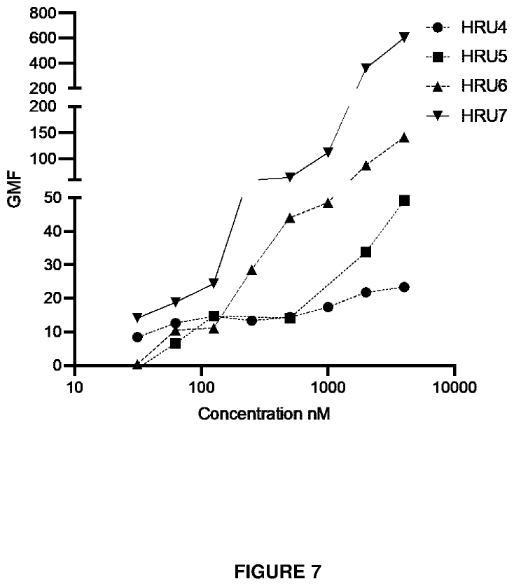

Mouse platelets were labeled in vivo with anti CD42c-Dylight649 antibody. The animals were treated with the HIT-like monoclonal antibody KKO plus heparin in the absence (vehicle) or presence of 3.49×10⁻¹¹ moles per gram (˜1 microgram/g) of parental scFv or HRU5. Fixed lungs from these mice were scanned with an IVIS Lumina Spectrum CT. There was clear accumulation of florescence in lungs treated with KKO plus vehicle, indicative of the presence of platelet rich thrombi. Treatment with parental scFv or HRU5 resulted in inhibition of thrombus accumulation (FIGS. 5A and B). Clot inhibition was much greater in mice treated with HRU5 than with parental scFv, indicating the increased activity of this new molecule against immune complex-induced thrombosis. This finding is consistent with the findings of the in vitro experiments and microfluidics studies. HRU6 demonstrated unexpectedly strong binding to monocytes (FIGS. 6A and B). The binding of HRU4, HRU5 and HRU6 to human platelets can be seen in FIG. 7. As expected, HRU5 showed strong binding to platelets due to the presence of the functional linker. HRU6 and HRU7 demonstrated stronger binding to human platelets than expected.

### SUMMARY

This Example demonstrates that scFvs derived from the IV.3 monoclonal antibody have been improved by the addition of a functional linker between the variable heavy and variable light chains that increases binding affinity to FcγRIIA. Addition of anti-thrombin moieties such as lepirudin and bivalirudin led to an unexpected significant increase in binding to platelets and monocytes, and enhanced functional activity. Therefore, these molecules more effectively inhibit immune-complex induced thrombosis.

HRU5 to HRU7 are effective FcγRIIA blocking agents. In addition, HRU6 and HRU7 possess enhanced binding affinity for FcγRIIA and anticoagulant function.

### Example Two: Envisaged Treatment of Vaccine-Induced Immune Thrombotic Thrombocytopenia (VITT) Using the Antigen-Binding Fragments of the Invention

Example Two is a prophetic Example.

The skilled person can use the directions provided in this Example to test the efficacy of HRU5, HRU6 and/or HRU7 in treating VITT.

Materials and Methods

Patient Samples

Patients will be selected with laboratory test results and clinical features consistent with those of previously reported cases of VITT. Patients will have received at least their first dose of a COVID-19 adenoviral vector vaccine (for example, Vaxzevria [ChAdOx1 nCoV-19], AstraZeneca; University of Oxford) before the onset of symptoms, had thrombocytopenia, high levels of D-dimer and anti PF4 antibodies detected by enzyme-linked immunosorbent assay. Patient samples will also be positive for platelet activation functional assays.

FcγRIIA/hPF4 Double Transgenic Mice

FcγRIIA/hPF4 double transgenic mice can be used in these experiments, and for example may be those described in Reilly et al., “Heparin-induced thrombocytopenia/thrombosis in a transgenic mouse model requires human platelet factor 4 and platelet activation through FcγRIIA” Blood. 2001; 98(8):2442-2447 and in Section (ix) of the “Materials and methods” section of Example One. The animals can, for example, be injected intravenously (iv) with IgG from the VITT patients.

HRU Molecules

HRU molecules can, for example, be prepared according to the methods described in Sections (ii) and (iii) of the “Materials and methods” section of Example One and can be injected into the mice described above via the iv route. Lungs can be harvested approximately 4 h after treatment, fixed in formalin and scanned for fluorescence in an IVIS Lumina Spectrum CT Spectrometer (PerkinElmer).

Expected Results

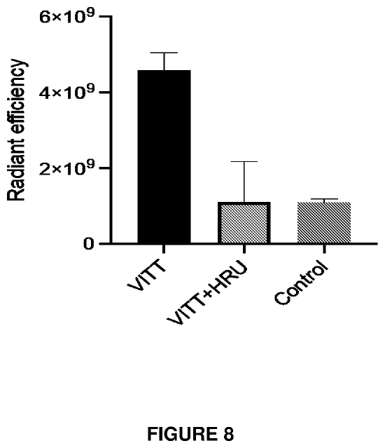

Antibodies from the VITT patients are expected to induce thrombosis in the double transgenic mice. Thrombosis is expected to be strongly inhibited by administration of HRU4 (at least to levels shown in FIG. 8). Administration of HRU5, HRU6 and/or HRU7 in treating VITT is expected to produce results at least equivalent to those shown for HRU4 in FIG. 8 and it is anticipated that the results for HRU5, HRU6 and/or HRU7 may exceed the results observed for HRU4. Results for HRU5, HRU6 and/or HRU7 can be assessed using the methods described in Sections (vi)-(ix) of the “Materials and methods” section of Example One.

### Example Three: Comparing the Effect of the FcγRIIA Binding Peptide to that of HRU5

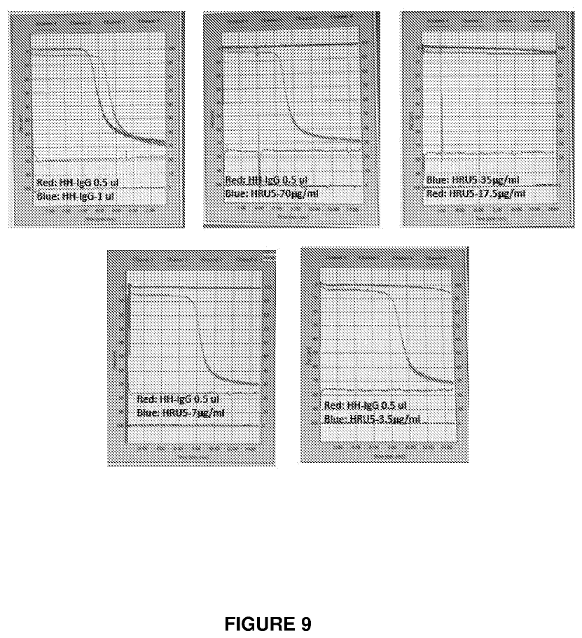

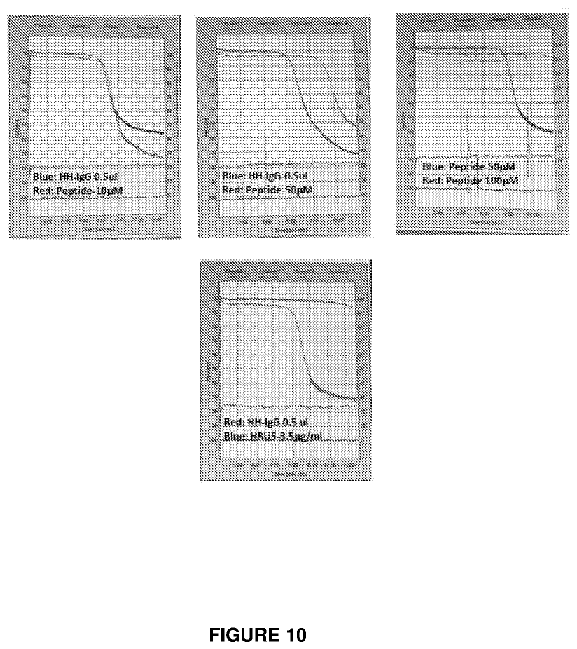

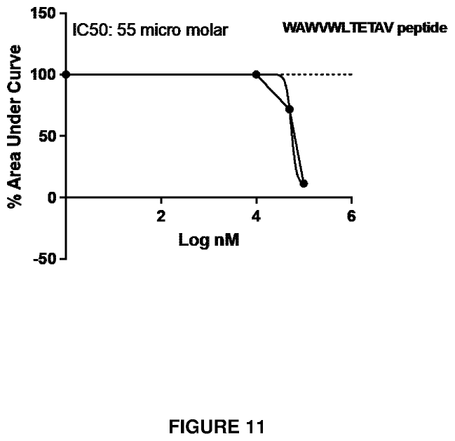

The IC50 of the FcγRIIA binding peptide (SEQ ID NO: 11) and HRU5 were calculated by inhibiting platelet aggregation induced by serum from a HIT patient in the same manner as described in Section (v) of the “Materials and methods” section of Example One. Raw data used to calculate the IC50 of the FcγRIIA binding peptide and HRU5 is provided in FIG. 9. Inhibition of platelet aggregation of HIT-antibody by HRU5 ˜3.5 mg/ml. A comparison of the effect on platelet aggregation of the FcγRIIA binding peptide and HRU5 is provided in FIG. 10, which shows that inhibition of platelet aggregation of HIT-antibody by HRU5 ˜3.5 mg/ml.=˜93.86 nM, whereas inhibition of platelet aggregation of HIT-antibody by the FcgRIIA binding peptide was at 100 μM. FIG. 10 clearly shows that the capacity of the FcgRIIA binding peptide alone to inhibit HIT serum-induced aggregation is low. The IC50 for the FcgRIIA binding peptide alone is shown in FIG. 11.

Expected Peptide Binding Contribution

Contribution of the FcgRIIA binding peptide to the molecular weight of HRU5:

The molecular weight of HRU5 is 28.5 kDa. The contribution of the peptide is 1.36 kDa or 4.7% of HRU5.

As the IC50 of the FcgRIIA binding peptide alone is 55,000 nM, (i.e 3,000 times lower than the EC50 of the parental scFv), its impact on binding is expected to be negligible at the concentrations used.

Therefore, the significant increase in activity observed in HRU5 relative to the parental scFv (55,000 nM to 8.8 nM) is not expected from the presence of the peptide alone. It was completely unexpected that the addition of the peptide would lead to a significant increase in activity at a much lower effective concentration. The addition of a weak binding peptide led to an unexpected large increase in activity. The addition of the functional peptide/linker creates a surprising synergistic effect which is more than an additive effect.

### Example Four: Influence of the Location of the FcγRIIA Binding Peptide on its Activity

This Example demonstrates that the location of the FcgRIIA binding peptide is not random and determines its capacity to influence activity.

C1 Construct

A C1 construct was created consisting of the parental scFv (the humanized scFv used in Example One) with the FcgRIIA binding peptide (SEQ ID NO: 11) added to the N terminus of the parental scFv (shown in bold in the sequence below).

SEQ ID NO 20:
MKSLITPITA GLLLALSQPL LAWAWVWLTE TAVGGGGEVQ
LVESGPELKK PGETVKISCK ASGYTFTNYG MNWVKQAPGK
GLKWMGWLNT YTGESIYPDD FKGRFAFSLE TSASTAYLQI
NNLKSEDTAT YFCARGDYGY DDPLDYWGQG TSVTVSSGGG
GSGGGGSGGG GSDIVMTQAP PSVPVTPGES VSISCRSSKS
LLHTNGNTYL HWFLQKPGQS PRLLIYRMSV LASGVPDRES
GSGSGTDFTL KISRVEAEDV GVYYCMQHLE YPLTFGAGTK
LEIKRAEQKL ISEEDLHHHH HH

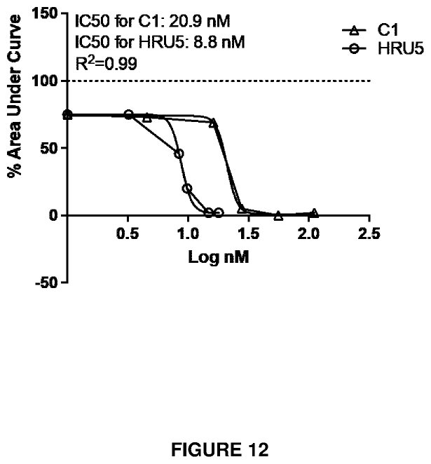

Addition of the peptide at the N terminus did not enhance the activity of the parental scFv (EC50 for parental scFv 18 nM; EC50 for C1 construct 20.9 nM) (FIG. 12). Addition of the peptide at the N terminus increased the EC of the parental scFv. Therefore, addition of the peptide to the N terminus of the scFv is ineffective.

The results obtained in Example Four are surprising as it is conventional to add a secondary peptide to the N-terminus of the parent molecule (the scFV in this case) because it avoids disrupting the conformation or shape of the parent, hence avoiding disrupting its function. However, no increase in function was observed using the conventional approach.

However, when the FcgRIIA binding peptide was inserted in the middle of the parent molecule (in the linker region), which is an unconventional approach as there is a risk of disruption of its confirmation and hence its function, instead of a decrease in function, an unexpected large enhancement of function was observed.

Molecular Modelling

SWISS-MODEL (https://swissmodel.expasy.org/) from the Swiss Institute of Bioinformatics was used to model the protein structure (homology-modelling) of the parental scFv (HRU4) and HRU5.

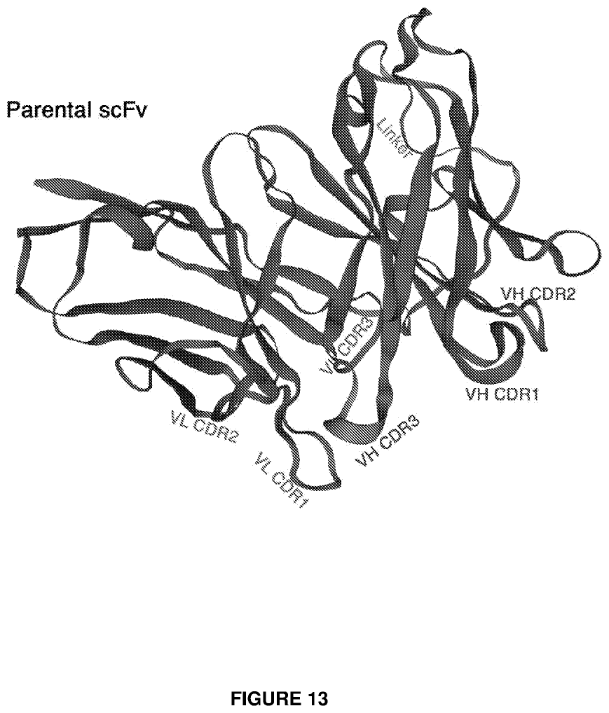

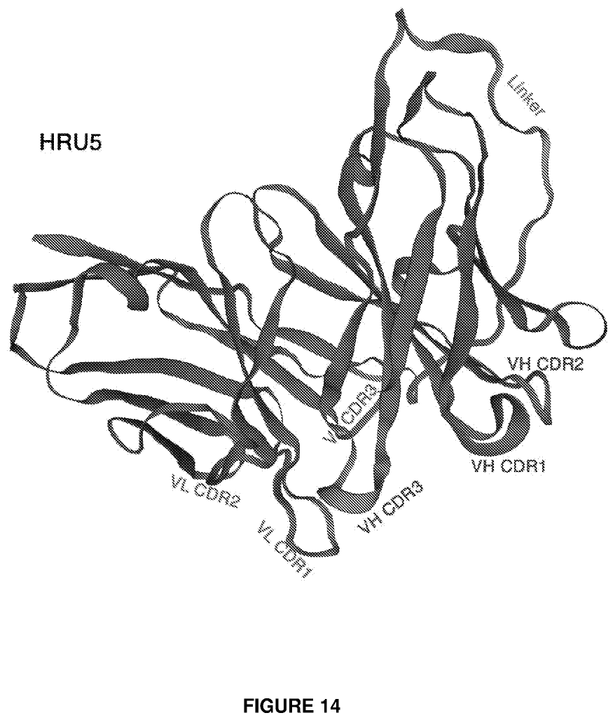

The Figures show the predicted structure of parental scFv (FIG. 13) and HRU5 (FIG. 14). The position of the CDRs and linkers are indicated. HRU6 couldn't be modelled because there is no existing crystal or NMR structure of lepirudin or herudin. However, modelling of HRU6 would result in the same structure as HRU5.

The flexible linker in the parental scFv is buried within the structure and only provides a linking function between VH and VL domains (FIG. 13).

The functional linker (flexible linker plus peptide), on the other hand, is accessible and provides a separate binding surface away from the CDRs (FIG. 14).

There was no a priori reason to think that adding the peptide to the flexible linker would enhance binding and activity, since the linker may not have been accessible to the antigen. Hence it may not have necessarily worked. The structure presented in FIG. 14 suggests that it may work due its position away from the main scFv structure.

Flexible linkers are desired in scFvs as they serve as mere connectors between the antibody domains and do not interact with the scFv. Hence, addition of a non-flexible linker, like the one used in HRU5-7, was likely to produce a decrease in binding activity of the scFv.

Expression in E. coli

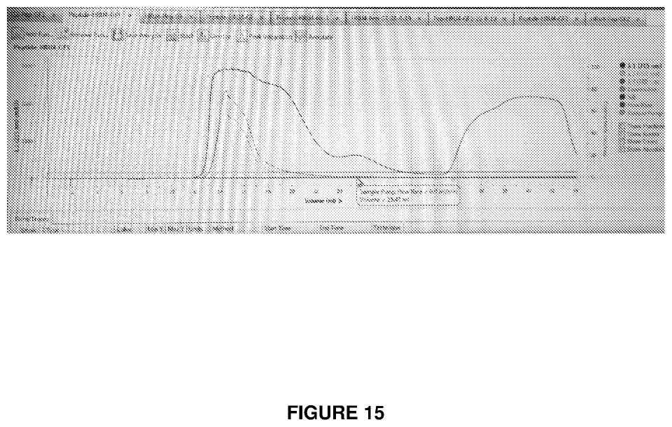

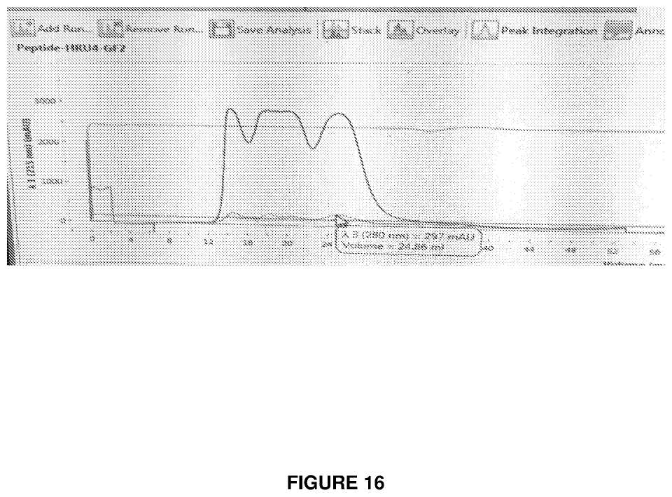

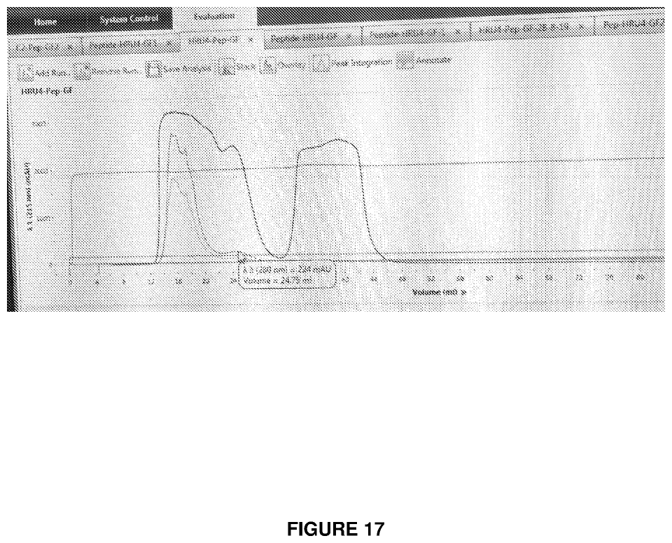

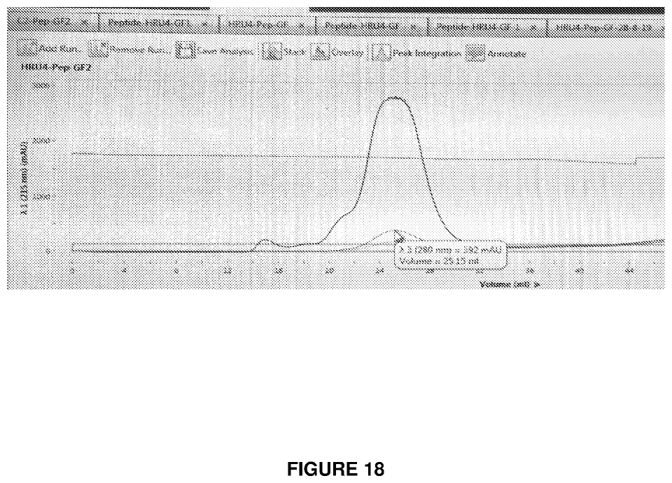

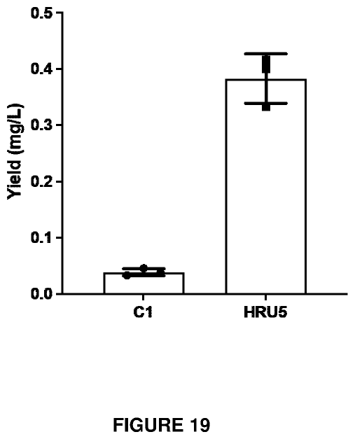

The constructs were expressed and purified as described Section (iii) of the “Materials and methods” section of Example One. Protein concentration was determined by spectrometry (Direct Detect Spectrometer). The yield is expressed in mg of purified protein per litre of bacterial culture. The expression of HRU5 was 10× higher than the expression of C1 (Table 2 and FIGS. 15-19)

TABLE 2
Expression of C1 and HRU5 in E. coli

| Date | C1 | HRU5 | |
|---|---|---|---|
| 29 Apr. 2019 | | 1 mg/3 L | |
| 9 May 2019 | 0.45 mg/12 L | | |
| 18 Jun. 2019 | 1.1 mg/24 L | | |
| 21 Jun. 2019 | | 1.25 mg/3 L | |
| 28 Sep. 2019 | 0.4 mg/12 L | | |
| 9 Jul. 2020 | | 1.2/3 | L |
| Mean/L Culture of Bacteria | 0.039 mg | 0.383 | mg |

These results suggest that the antigen-binding fragments of the invention will be easy to manufacture.

### Example Five: Envisaged Testing of Different Positions for the FcgRIIA Binding Peptide

Example Five is a prophetic Example.

The skilled person can use the directions provided in this Example to test the efficacy of antigen-binding fragments in which the FcgRIIA binding peptide has been placed in different positions.

Whilst the position of the FcgRIIA binding peptide has been tested in one position in the linker region in the present application, it will become immediately obvious to the skilled person that the binding peptide could potentially be placed 1, 2, 3, 4, 5, 6 or up to 28 amino acid positions to the left or right of the position used in the present application without disrupting the binding affinity of the CDRs.

The skilled person could use the Examples provided in in Sections (ii-ix) of the “Materials and methods” section of Example One to create antigen-binding fragments with the FcgRIIA binding peptide in various positions and test their efficacy.

Expected Results

It is envisaged that antigen-binding fragments with the FcgRIIA binding peptide placed 1, 2, 3, 4, 5, 6 or up to 28 amino acid positions to the left or right of the position used in the present application may also provide the beneficial effects of the invention. In some embodiments it could be expected that such antigen-binding fragments would achieve the beneficial effects of the invention by allowing the functional linker to be accessible and/or by providing a separate binding surface away from the CDRs. Antigen-binding fragments with the FcgRIIA binding peptide 1, 2, 3, 4, 5 or 6 amino acid positions to the left or right of the position used in the present application may provide the benefits of the invention as the functional linker would remain within the linker region.

1. An antigen-binding fragment that specifically binds FcγRIIA, wherein the antigen-binding fragment comprises a heavy chain variable region, a light chain variable region and a linker, and wherein at least a portion of the linker binds FcγRIIA.

2. The antigen-binding fragment according to claim 1, wherein the heavy chain variable region and the light chain variable region are joined by the linker.

3. The antigen-binding fragment according to claim 1 or claim 2, wherein the antigen-binding fragment comprises:
a heavy chain variable region comprising:
a) a heavy chain CDR1 with an amino acid sequence according to SEQ ID NO: 4;
b) a heavy chain CDR2 with an amino acid sequence according to SEQ ID NO: 5;
c) a heavy chain CDR3 with an amino acid sequence according to SEQ ID NO: 6;
and a light chain variable region comprising:
d) a light chain CDR1 with an amino acid sequence according to SEQ ID NO: 7;
e) a light chain CDR2 with an amino acid sequence according to SEQ ID NO: 8; and
f) a light chain CDR3 with an amino acid sequence according to SEQ ID NO: 9.

4. The antigen-binding fragment according to any one of claims 1 to 3, wherein the linker comprises the amino acid sequence:
WAW X₁ W X₂ TET X₃ V
and wherein:
X₁ is selected from V or A;
X₂ is selected from L or A; and
X₃ is selected from A or G.

5. The antigen-binding fragment according to any one of claims 1 to 4, wherein the linker comprises an amino acid sequence with at least 90% sequence identity to SEQ ID NO: 11.

6. The antigen-binding fragment according to any one of claims 1 to 5, wherein the linker comprises an amino acid sequence according to SEQ ID NO: 11.

7. The antigen-binding fragment according to any one of claims 1 to 6, wherein the heavy chain variable region comprises an amino acid sequence with at least 90% sequence identity to SEQ ID NO: 15.

8. The antigen-binding fragment according to any one of claims 1 to 7, wherein the light chain variable region comprises an amino acid sequence with at least 90% sequence identity to SEQ ID NO: 16.

9. The antigen-binding fragment according to any one of claims 1 to 8, wherein:
a) the heavy chain variable region comprises an amino acid sequence according to SEQ ID NO:15, or a variant of that sequence having 1, 2, or 3 amino acid substitutions in the framework region; and/or
b) the light chain variable region comprises an amino acid sequence according to SEQ ID NO:16, or a variant of that sequence having 1, 2, or 3 amino acid substitutions in the framework region.

10. The antigen-binding fragment according to any one of claims 1 to 9, wherein the position of the functional linker is:
a) within a heavy and/or light chain but not within a CDR; or
b) 1, 2, 3, 4, 5, 6, 7, 8, 9, 10 or more amino acid positions to the left or right of the position according to SEQ ID NO:3.

11. The antigen-binding fragment according to any one of claims 1 to 10, wherein the functional linker is not located at the N-terminus or the C-terminus of the antigen-binding fragment.

12. The antigen-binding fragment according to any one of claims 1 to 11, wherein the functional linker is positioned so that it is partially or completely exposed on the outside of the tertiary structure of the antigen-binding fragment.

13. The antigen-binding fragment according to any one of claims 1 to 12, wherein the functional linker is positioned so that it enhances the capacity of the antigen-binding fragment to bind to FcγRIIA.

14. The antigen-binding fragment according to any one of one of claims 1 to 13, wherein the antigen-binding fragment is conjugated to an anticoagulant.

15. The antigen-binding fragment according to claim 14, wherein the anticoagulant is selected from the group consisting of danaparoid, desirudin, tick anticoagulant peptide, factor Xa inhibitors, prothrombin inhibitors, tissue factor inhibitors, FXII inhibitors, danaparoid, bivalirudin, lepirudin, argatroban, and any combination thereof.

16. The antigen-binding fragment according to claim 13 or claim 14, wherein the anticoagulant is bivalirudin and/or lepirudin.

17. The antigen-binding fragment according to claim 16, wherein the antigen-binding fragment comprises an amino acid sequence according to SEQ ID NO:12, or a variant of that sequence having 1, 2, or 3 amino acid substitutions in the framework region.

18. The antigen-binding fragment according to claim 16, wherein the antigen-binding fragment comprises an amino acid sequence according to SEQ ID NO:14, or a variant of that sequence having 1, 2, or 3 amino acid substitutions in the framework region.

19. The antigen-binding fragment according to any one of claims 1 to 18, wherein the antigen-binding fragment is a single-chain variable fragment (scFv).

20. The antigen-binding fragment according to any one of claims 1 to 19, wherein the functional linker is positioned adjacent to or within a flexible linker.

21. The antigen-binding fragment according to claim 20, wherein the flexible linker comprises or consists of neutral amino acids.

22. The antigen-binding fragment according to claim 21, wherein the flexible linker comprises or consists of between 1 and 15 amino acids, between 3 and 14 amino acids, between 3 and 14 amino acids, between 9 and 13 amino acids, between 10 and 12 amino acids, or 11 amino acids.

23. A nucleic acid molecule encoding the antigen-binding fragment according to any one of claims 1-22.

24. A vector comprising the nucleic acid molecule according to claim 23.

25. A host cell comprising the vector according to claim 24.

26. The host cell according to claim 25, wherein the host cell is derived from a mammal, insect, plant or microbe.

27. A pharmaceutical composition comprising the anti-binding fragment of any one of claims 1-22.

28. A method of treating a subject with a thrombogenic-related disease, the method comprising administering to the subject a therapeutically effective amount of the antigen-binding fragment of any one of claims 1-22 or the pharmaceutical composition of claim 27.

29. Use of the antigen-binding fragment of any one of claims 1-22 in the manufacture of a medicament for treating a thrombogenic-related disease in a subject in need thereof.

30. The antigen-binding fragment of any one of claims 1-22 for use in the treatment of a thrombogenic-related disease in a subject in need thereof.

31. The method according to claim 28, the use according to claim 29 or the antigen-binding fragment according to claim 30, wherein the thrombogenic-related disease is heparin-induced thrombocytopenia (HIT), immune thrombocytopenia (ITP), an immune platelet disorder with associated thrombosis, NETs-induced thrombo-embolism, organ injury, a NETs-associated disorder, drug-induced ITP, viral infection (e.g. SARS infection, COVID-19 infection), bacterial infection, fungal infection, parasitic infection, sepsis, antibody-induced ITP, antiphospholipid syndrome, cancer-induced thrombocytopenia, thrombo-embolism, an autoimmune or inflammatory disease involving CD32 (including rheumatoid arthritis, osteoarthritis, systemic lupus erythematosus and psoriasis), or a disorder or disease mediated by CD32 involving one or more of the following cells: platelets, neutrophils, monocytes, macrophages, eosinophils, basophils and mast cells.

32. The method, use or antigen-binding fragment according to claim 31, wherein the thrombogenic-related disease involves the binding of immune complexes to FcγRIIA.

33. The method, use or antigen-binding fragment according to claim 31 or claim 32, wherein the thrombogenic-related disease is heparin-induced thrombocytopenia (HIT).

34. The method, use or antigen-binding fragment according to claim 31 or claim 32, wherein the ITP is primary ITP with associated thrombosis.

35. The method, use or antigen-binding fragment according to claim 31 or claim 32, wherein the ITP is secondary ITP.

36. The method, use or antigen-binding fragment according to claim 35, wherein the secondary ITP is secondary ITP with associated anti-phospholipid antibody syndrome, systemic lupus erythematosus, Evans syndrome or chronic infection.

37. A method of treating a subject with a disease related to FcγRIIa-mediated neutrophil activation, the method comprising administering to the subject a therapeutically effective amount of the antigen-binding fragment of any one of claims 1-22 or the pharmaceutical composition of claim 27.

38. Use of the antigen-binding fragment of any one of claims 1-22 in the manufacture of a medicament for treating a disease related to FcγRIIa-mediated neutrophil activation in a subject in need thereof.

39. The antigen-binding fragment of any one of claims 1-22 for use in the treatment of a disease related to FcγRIIa-mediated neutrophil activation in a subject in need thereof.

40. The method according to any one of claims 28 or 31 to 37, the use according to any one of claim 29, 31 to 36 or 38, or the antigen-binding fragment according to any one of claim 30 to 36 or 39, wherein the antigen-binding fragment is administered by a route selected from the group consisting of intravenous, intramuscular, subcutaneous, intraperitoneal, or any combination thereof.

41. The method according to any one of claim 28, 31 to 37 or 40, the use according to any one of claim 29, 31 to 36, 38 or 40, or the antigen-binding fragment according to any one of claims 30 to 36 or 39 to 40, wherein the amount of antigen-binding fragment administered is from about 5 mg/kg to about 50 mg/kg, or the amount of antigen-binding fragment administered is via intravenous infusion at a dosage of about 0.1 mg/kg/hr to about 0.5 mg/kg/hr, about 0.1 mg/kg/hr to about 1 mg/kg/hr, about 0.5 mg/kg/hr to about 5 mg/kg/hr, or at about 5 mg/kg/hr to ab out 1 Omg/kg/hr.

42. The method according to any one of claims 28, 31 to 37 or 40 to 41, the use according to any one of claims 29, 31 to 36, 38 or 40 to 41, or the antigen-binding fragment according to any one of claims 30 to 36 or 39 to 41, wherein the subject is human.
